(19) United States
(12) Patent Application Publication
(10) Pub. No.: US 2024/0110143 A1
(43) Pub. Date: Apr. 4, 2024

(54) DYNAMICALLY INTERCONNECTED MICROBIOREACTORS AND APPLICATIONS THEREOF

(71) Applicant: VANDERBILT UNIVERSITY, Nashville, TN (US)

(72) Inventors: John P. Wikswo, Brentwood, TN (US); Kieran David Nehil-Puleo, Nashville, TN (US); Ronald S. Reiserer, Nashville, TN (US)

(21) Appl. No.: 18/527,751

(22) Filed: Dec. 4, 2023

Related U.S. Application Data
(63) Continuation-in-part of application No. 17/947,302, filed on Sep. 19, 2022.
Application No. 17/947,302 is a continuation of application No. 17/578,966, filed on Jan. 19, 2022, now Pat. No. 11,447,734.
Application No. 17/578,966 is a continuation-in-part of application No. PCT/US2021/042179, filed on Jul. 19, 2021.
Application No. 17/578,966 is a continuation-in-part of application No. PCT/US2020/040061, filed on Jun. 29, 2020.
(60) Provisional application No. 63/429,680, filed on Dec. 2, 2022.
(60) Provisional application No. 63/139,138, filed on Jan. 19, 2021.
(60) Provisional application No. 63/163,160, filed on Mar. 19, 2021.
(60) Provisional application No. 63/257,149, filed on Oct. 19, 2021.
(60) Provisional application No. 63/277,329, filed on Nov. 9, 2021.
(60) Provisional application No. 63/300,321, filed on Jan. 18, 2022.
(60) Provisional application No. 63/053,388, filed on Jul. 17, 2020.
(60) Provisional application No. 63/139,138, filed on Jan. 19, 2021.
(60) Provisional application No. 63/163,160, filed on Mar. 19, 2021.
(60) Provisional application No. 62/868,303, filed on Jun. 28, 2019.

(51) Int. Cl. C12M 1/00; C12M 1/06; C12M 1/22; C12M 1/24; C12M 1/32; C12M 1/36; C12M 1/42; C12M 3/04; C12M 3/06

(52) U.S. Cl. CPC C12M 29/10; C12M 23/08; C12M 23/10; C12M 23/12; C12M 23/16; C12M 23/50; C12M 23/58; C12M 27/02; C12M 27/12; C12M 35/02; C12M 35/04; C12M 41/48

(57) ABSTRACT

One aspect of the invention provides a network platform that includes a fluidic network comprising one or more pumps, and one or more valves, and a plurality of fluidic modules interconnected by the fluidic network of the one or more pumps and the one or more valves to allow controlled transfer of suspended cells, other substances, and fluids from one fluidic module to another, or self-circulation.

### CROSS-REFERENCE TO RELATED PATENT APPLICATIONS

This application claims priority to and the benefit of U.S. Provisional Patent Application Ser. No. 63/429,680, filed Dec. 2, 2023.

This application is also a continuation-in-part application of U.S. patent application Ser. No. 17/947,302, filed Sep. 19, 2022, which is a continuation application of U.S. patent application Ser. No. 17/578,966, filed Jan. 19, 2022, now U.S. Pat. No. 11,447,734, which itself claims priority to and the benefit of U.S. Provisional Patent Application Serial Nos. 63/139,138, filed Jan. 19, 2021, 63/163,160, filed Mar. 19, 2021, 63/257,149, filed Oct. 19, 2021, 63/277,329, filed Nov. 9, 2021, and 63/300,321, filed Jan. 18, 2022.

This application is also a continuation-in-part application of U.S. patent application Ser. No. 18/015,782, filed Jan. 12, 2023, which is a U.S. national entry of PCT Patent Application Serial No. PCT/US2021/042179, filed Jul. 19, 2021, which itself claims priority to and the benefit of U.S. Provisional Patent Application Serial Nos. 63/053,388, filed Jul. 17, 2020; 63/139,138, filed Jan. 19, 2021; and 63/163,160, filed Mar. 19, 2021.

Each of the above-identified applications is incorporated herein by reference in its entirety.

### STATEMENT AS TO RIGHTS UNDER FEDERALLY-SPONSORED RESEARCH

This invention was made with government support under Grant No. 2117782 awarded by the National Science Foundation (NSF). The government has certain rights in the invention.

### FIELD OF THE INVENTION

The invention relates generally to bioreactors, and more particularly to dynamically interconnected microbioreactors and applications thereof.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

One of the important lessons of the COVID-19 pandemic is that rapid translation of experimental medicines from the laboratory to treatments for the entire human population requires not only enormous funding, but also the technological means for the pharmaceutical and biologics industries to mass-manufacture said treatments. Another is the realization that we will undoubtedly face pandemic-level challenges in the future. To combat the inevitable emerging superbugs, we must improve our ability to research, characterize, and scale up the production of experimental medicines, and reduce the time from identification of a new infectious agent to the widespread availability of the appropriate vaccines and other pharmaceuticals.

More generally, it is clear that a growing fraction of drugs introduced and produced by the pharmaceutical industry are biomolecules produced by microbial, mammalian, and other cells cultured in industrial-scale bioreactors. The chemical, energy, and food industries are increasingly introducing new products and technologies that rely on living cells cultured in bioreactors. In each of these cases, there is a pressing need for technologies that support the quantitative determination of the factors that limit the scale-up of cell culture technologies from nanoliter microfluidic devices and micro- and milliliter well plates all the way to production bioreactors with volumes of thousands to a hundred thousand liters.

A breadth of industries uses living cellular systems to synthesize pharmaceuticals, industrial chemicals, food, and other products. This biologics industry, which includes what is also known as biopharma, is concerned with the fabrication of complex, biologically derived products, typically produced using heavily engineered strains of microorganisms. Biologics cover a wide range of products such as vaccines, allergenics, extracellular vesicles, mesenchymal stromal cells, somatic cells, tissues, and recombinant therapeutic proteins. Biologics relevant during the era of COVID-19 are monoclonal antibodies, which can be produced by the genetic engineering of Chinese Hamster Ovarian (CHO) cells to produce humanoid, recombinant proteins, i.e., antibodies. To produce biologics, the industry uses several organisms, including mammalian cells such as CHO cells, bacteria such as E. coli, and fungi such as the yeast S. cerevisiae. To produce the massive quantities of biologics in demand, industrial-scale bioreactors containing thousands or even tens of-thousands of liters of liquid growth media are used to grow cells. The large volume of the bioreactors allows the total population of the microorganism colony to be increased, thereby increasing the total amount of a biologic the bioreactor can produce. Along with growing these microorganisms in large-scale bioreactors, the strains are highly optimized for production to further increase yields.

The biologics industry is enormous and impacts several domains of the world economy: in 2022, the global biologics industry was valued at $382 billion, with a projected compound annual growth rate of 12.9%, making it one of the largest and fastest growing industries on the planet. The large growth of the biologics industry indicates that in the coming years both the demand for and ubiquity of biological products will increase. To address demand and meet the urgent need to counteract the rapid deterioration of many current anti-pathogenic treatments by creating new ones, innovations that expedite the transfer of biotechnology products from the laboratory to production are necessary. Specifically, there is a need in the biotechnology and biologics industries for instruments that are capable of growing and characterizing cells possessing as-yet unspecified genes.

Factors Hindering Biologic Production: (1) Strain Engineering. Strain engineering for biologic production involves discovering and engineering the genetics of an organism to create a strain that maximally produces some bioproduct of interest. The intensive selection of strains is necessary because of the significant difference in productivity from strain to strain within a species. Due to the unknown function and large number of genes in most organisms used for biologic production, many of the genetic changes made to an organism are observational: a gene is changed in the organism and then its phenotypic response is characterized to assess whether the change was beneficial. Given the large number of experiments and other variables, strain engineering is a bottleneck in the development of new biologics.

(2) Research Transferability. Another major obstacle inherent to the current methods we use to identify highly producing strains is uncertainty that the results of research conducted under laboratory conditions will transfer when the organisms are grown under industrial conditions. Industrial bioreactors are known to contain heterogeneous environmental conditions which, if the strain is sufficiently sensitive, have been shown to be capable of reducing the productivity, or in the worst case, even killing the colony of organisms. As cells circulate through a bioreactor, even brief exposures to high or low levels of oxygen, pH, nutrients, or metabolites can lead to long-term changes in the expression of particular genes in a manner that reduces the average production rate of the briefly exposed cells. Identification of the genes with excessive environmental sensitivities can in some cases be addressed by deleting, suppressing, or replacing those genes. However, the required first step is to quantify the cellular sensitivity and identify the genes. This may be difficult in experiments run in only one or more small bioreactors whose conditions cannot be changed as rapidly as the fluctuations experienced by cells in much larger continuous stirred bioreactor.

Industrial and laboratory bioreactors differ by several orders of magnitude in terms of volume and cell population: industrial bioreactors can be on the order of 10⁵ liters whereas laboratory bioreactors are typically <1 liter, with some of the smallest commercial systems having volumes of 15 ml. Due to their large volume, industrial-scale reactors possess more heterogeneous environmental conditions than those seen in small-scale reactors. This difference in volume results in differences in the homogeneity of the growth conditions within each reactor. In large-scale bioreactors with large volumes of liquid, the ability to homogeneously mix the cell-media-metabolite slurry is reduced, resulting in spatiotemporal heterogeneities of liquid/cellular shearing and reduced mass transfer of nutrients, waste products, and critical metabolites and secreted cell products. Cells are often sensitive to changes in environmental conditions and therefore require growth conditions as close to optimal as possible to achieve maximal productivity. This sensitivity to conditions is particularly significant for mammalian cells, which can be easily damaged by high shear stresses and, counter-intuitively, also low shear stresses. Because of the heterogeneity in conditions in industrial bioreactors, cells are more likely to under-perform as compared to what was demonstrated in small-scale bioreactors.

(3) Reproduction of Large-Scale Conditions. The ideal method to develop strains that are resistant to heterogeneities would be to characterize fully the performance of strains using industrial bioreactors, so that the conditions of characterization would be identical to those of production, resulting in a high confidence that the developed strains would retain productivity. Industrial-scale bioreactors are expensive to purchase, maintain, and operate, however, and are therefore uneconomical to use for strain characterization, where one might wish to compare dozens or even hundreds of different strains under a wide variety of media and growth conditions over long periods of time.

On both the large and small scales, the most commonly used bioreactors are the stirred-tank reactor (STR), which is run in batch mode with an initial delivery of nutrient-rich media, and the continuous stirred-tank reactor (CSTR), in which there is a continuous delivery of media. In stirred reactors, the mixing of the cell-media-metabolite slurry within the bioreactor is performed using an impeller to physically stir the liquid and cells. On the small scale, a slower radial velocity is needed to mix the slurry, possibly achieved with a stir bar or shaker, such that issues with shear stress damaging the cells sometimes arise only when the scale of the bioreactor and the impeller velocity are increased.

Strain discovery and media optimization are resource- and time-intensive processes that are typically mandatory for any new biologic product. The problem of heterogeneity within large reactors presents a systemic challenge to the discovery of new strains and the optimization of growth conditions, since for reasons of economics and efficiency, strain engineering is typically conducted in laboratories with as few cells and as small a media volume as possible. Results obtained using small-scale reactors often fail to reproduce the conditions of industrial production. The unaddressed discrepancy in conditions between industrial and laboratory bioreactors when conducting research can result in unexpected drops in the performance of a strain when the bioreactor size is scaled up to the industrial level, with serious economic consequences and lost time-to-market.

The resulting low confidence in the viable scaling of strains from laboratory to industrial production is a foundational problem since strain discovery is a resource- and time-intensive process and is often mandatory for any new biologic product. To increase the confidence that strains researched in the laboratory will retain their productivity, the development of a flexible, quantitative, and accurate model of industrial bioreactors is required.

Therefore, a heretofore unaddressed need exists in the art to address the aforementioned deficiencies and inadequacies.

### SUMMARY OF THE INVENTION

One aspect of the present invention relates to a network platform comprising a fluidic network comprising one or more pumps, and one or more valves; and a plurality of fluidic modules interconnected by the fluidic network of the one or more pumps and the one or more valves to allow controlled transfer of suspended cells and fluids from one fluidic module to another, or self-circulating.

In one embodiment, the network platform further comprises at least one input media reservoir and/or at least one collection reservoir in fluidic communication with the fluidic network for providing inputs and/or collecting outputs of any one of the plurality of fluidic modules, respectively.

In one embodiment, the plurality of fluidic modules comprises bioreactors, wells, organs-on-chips, chemostats, or a combination of them.

In one embodiment, each of the plurality of fluidic modules is individually perfusable.

In one embodiment, rate of perfusion of each individually perfusable fluidic module is controlled by at least one of the one or more pumps and the one or more valves.

In one embodiment, the one or more valves are a rotary planar valve system that is operably regulated with a single motor as a time-domain fluidic multiplexer to move samples between each and every one of the plurality of fluidic modules.

In one embodiment, the one or more valves comprise an N×M crossbar valve that operably connects the at least one input media reservoir to the inputs of any one of the plurality of fluidic modules, and the outputs of the plurality of bioreactors to either the input of one of the bioreactors or the at least one collection reservoir, wherein each of N and M is an integer greater than zero.

In one embodiment, the one or more valves comprise n two-state crossbar valves, thereby creating 2ⁿ possible valve states, wherein n is an integer greater than zero.

In one embodiment, the fluidic network is a continuously pumped fluidic network configured to ensure that living cells are in tubes for only very short intervals of time, by being moved directly from one bioreactor to another without intermediate storage.

In one embodiment, the one or more pumps and the one or more valves are configured to ensure that all fluid lines are promptly washed to avoid the trapping or storage of cells in sub-optimal environments.

In one embodiment, the fluidic network further comprises a separating means coupled with the one or more pumps and the one or more valves for separating cells such that certain cells are recirculated to one fluidic module while others are allowed to be moved to another. In one embodiment, the separating means comprises a filter or other means to retain all cells within a fluidic module and only extract the fluid from one fluidic module for transfer to another.

In one embodiment, the separating means comprises a tangential flow filter, an alternating tangential flow filter, spiral cell separators, or other means.

In one embodiment, the fluidic network is a single large-scale crossbar valve system that operates with a single pneumatic, vertical via, rotary, or other mechanical valve at each intersection between every fluidic module inflow and outflow line, with a pump on each of either the inflow or outflow lines, or a dynamic multi-stage interconnection network that uses multiple smaller-scale crossbar or other valves.

In one embodiment, the fluidic network is a dynamically reconfigurable network.

In one embodiment, the interconnections of the plurality of fluidic modules are configured to create or simulate biological systems in which there are large-scale spatial gradients that support a variation in microbial composition.

In one embodiment, the interconnections of the plurality of fluidic modules are configured to allow any or all of the plurality of fluidic modules to connect to any other or all of the other fluidic modules.

In one embodiment, the plurality of fluidic modules is configured to serve as a physical but smaller scale model of the heterogeneous zonation within a larger reactor, and thereby to support the optimization of cell lines to ensure efficient bioproduction by cells upon scale-up.

In one embodiment, the plurality of fluidic modules comprises multiple microbioreactors that are linked together into a single combined bioreactor system to operably simulate the traversal of a cell through the different zonal conditions of the industrial bioreactor, creating a small-scale system that is able to model the conditions within an industrial-scale bioreactor.

In one embodiment, the plurality of fluidic modules comprises multiple microbioreactors configured to simulate spatiotemporal heterogeneities that are inherent in large-scale bioreactors, such that each microbioreactor represents a region or zone with a set of cell culture parameters including cell density, cell replication rate and division state, pH, shear stress, temperature mixing rates, and the concentration of nutrients, metabolites, oxygen, carbon dioxide, and other gases.

In one embodiment, the network platform is usable in combinatorial chemical processing, in which aliquots of different chemicals are combined or split.

In one embodiment, the network platform is usable in synthetic biology and/or DNA computing, in which aliquots of specifically coded RNA or DNA, or other molecular sequences are combined or separated.

These and other aspects of the invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIGS. 1A-1B show schematically the continuous distribution of conditions in a large-scale bioreactor (FIG. 1A) and how different regions in the bioreactor can be represented by smaller, serially connected bioreactors (FIG. 1B).

FIG. 2 is a more realistic but still simple representation of the key components in a stirred tank reactor (STR).

FIG. 3 shows a classical two-compartment scale-down system composed of a small continuous stirred reactor (CSR) and a plug flow reactor (PFR) connected in series.

FIGS. 4A-4C show how the continuous conditions of the bioreactor can be discretized into four zones of relative conditional equivalence (FIG. 4A), and how these zones can be represented by a combination of a CSR and a series or series-parallel PFR (FIGS. 4B-4C).

FIGS. 5A-5B show chemostats or perfusion bioreactors containing microbes such as yeast, with a pumped media source, stirring, oxygen delivery, and either passive (FIG. 5A) or active removal (FIG. 5B) of the excess media.

FIG. 6 shows a bidirectional microformulator that can move fluids between different bioreactors using valves and pumps that enable time-division multiplexing.

FIG. 7 shows four bioreactors permanently configured such that the effluent from four bioreactors is connected serially with pumps, valves, and other interconnects to emulate zones in a larger-scale bioreactor.

FIGS. 8A-8B show a generalized schematic that allows all possible connections between four bioreactors, hence creating a completely connected network of bioreactors (FIG. 8A) with a variety of active and passive interconnections (FIG. 8B).

FIGS. 9A-9I show a generic electrical crossbar in the “cross” (FIGS. 9A and 9E) and “bar” (FIGS. 9B and 9F) configurations and several means by which fluidic crossbar valves (FIGS. 9C, 9D, 9G and 9H-9I) can be implemented.

FIGS. 10A-10I show the eight states (FIGS. 10A-10H) of a blocking, 4×4 rotary planar crossbar valve (FIG. 10I).

FIGS. 11A-11F show how a set of 2×2 pneumatic or rotary planar crossbar switches can be used to interconnect an input media, four pumps, three bioreactors, and an output reservoir.

FIG. 12 shows a number of network topologies that can be implemented in microfluidics and are relevant to the problem of creating small-scale physical models of zonation in large bioreactors.

FIG. 13 shows a hypothetical network where the five zones of interest in the bottom row are connected not by simple pipes, but through a layer of reactor nodes that might serve the same training functions as does an intermediate layer in a neural net.

FIG. 14 shows a hypothetical network with five zones of interest in the bottom row and two intermediate layers of reactor nodes might serve the same training functions as do the multiple intermediate layers in a deeper neural net.

FIG. 15 shows a compact, non-blocking Bene network that could be used as an 8×8 crossbar valve, or even larger networks.

FIG. 16A shows one embodiment of an 8×8 simple grid network with a valve V element at each node.

FIGS. 16B-16D show 8×8 simple grid networks coupled to bioreactors B, pumps P, and reservoirs R to create fluid handing reactor networks that might be utilized in combinatorial chemical synthesis, synthetic biology, or DNA computing.

FIGS. 17A-17C show tape underlayment rotary node (TURN) valves that can be used to implement a crossbar valve system.

FIG. 18 shows how the TURN valve can be used in an array to create a microfluidic crossbar valve.

FIGS. 19A-19D show binary and continuously variable vertical via valves that can be used to implement a crossbar valve system.

FIG. 20 shows how vertical via valves can be used in an array to create a microfluidic crossbar valve.

FIG. 21 illustrates one compact implementation of the concepts underlying this invention for experimental study of the effects of bioreactor zonation.

### DETAILED DESCRIPTION OF THE INVENTION

The invention will now be described more fully hereinafter with reference to the accompanying drawings, in which exemplary embodiments of the invention are shown. The invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around,” “about,” “substantially” or “approximately” shall generally mean within 20 percent, preferably within 10 percent, and more preferably within 5 percent of a given value or range. Numerical quantities given herein are approximate, meaning that the terms “around,” “about,” “substantially” or “approximately” can be inferred if not expressly stated. As used herein, the terms “comprise” or “comprising,” “include” or “including,” “carry” or “carrying,” “has/have” or “having,” “contain” or “containing,” “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Developing complex networks is vital to understanding the physical zonation of large bioreactors. This invention can be easily reconfigured to investigate a multitude of zonation problems from small-scale reactors throughout scale-up to the largest reactors. Network analysis is used in many fields from communication networks to artificial neural networks and machine learning. Many of these networks can be useful for small-scale reactor modeling but can break down with bigger reactors or no longer provide meaningful representation of physical parameters within the larger reactor. Combinations of laminar and turbulent flow, as well as no slip boundaries and flow stagnation, are difficult to represent in simple node-edge models and require more complicated conditional and dependent connections.

This invention includes a set of cell culture bioreactors that are interconnected by pumps and valves to allow controlled transfer of suspended cells and fluids from one bioreactor to another. Simple single-pole/single-throw valves, two-by-two crossbar valves, and other small crossbar valves may no longer be appropriate when using small, interconnected bioreactors to guide the parameterization of digital twin models of real-world reactor scenarios. This invention allows rapid reconfiguration for control and experimentation by an artificial intelligence (AI) guided robot scientist that would allow simulation of various reactor configurations to be recapitulated both in silico and in vitro before large-scale investments for traditional scale-up processes.

A key application of this invention is to use multiple microbioreactors to simulate the spatiotemporal heterogeneities that are inherent in large-scale bioreactors, such that each microbioreactor represents a region or zone with a particular set of cell culture parameters, which include cell density, cell replication rate and division state, pH, shear stress, temperature mixing rates, and the concentration of nutrients, metabolites, oxygen, carbon dioxide, and other gases. The mass transfer between zones within the larger reactor would be simulated by the mass transfer between different microbioreactors as controlled dynamically by pumps and valves. In one embodiment, the pumps and valves are all integrated into the lid of a multi-well plate. The pumps and valves could include a means to separate cells such that certain cells were recirculated to the microbioreactor while others were allowed to be moved to another microbioreactor; this would include a filter or other means to retain all cells within a microbioreactor and only extract the fluid from one bioreactor for transfer to a second. The interconnection of multiple bioreactors can also be used to create or simulate biological systems in which there are large-scale spatial gradients that support a variation in microbial composition, as is the case with the microbiome in the human digestive track.

We present several embodiments of the system of interconnects that are based upon either a network of pumps and valves that allows any or all of the microbioreactors to connect to any other or all of the other microbioreactors, or time-domain fluidic multiplexers that can remove samples from one microbioreactor and add them to another. The network could be as simple as a single large-scale crossbar valve system that operates with a single pneumatic, vertical via, rotary, or other mechanical valve at each intersection between every bioreactor inflow and outflow line, with a pump on each of either the inflow or outflow lines, or a dynamic multi-stage interconnection network that uses multiple smaller-scale crossbar or other valves.

The network architecture for these multiple bioreactors can be used to create spatially distinct communities of microbes or microbial phenotypes, wherein intermediate network layers serve as a biological interface between different bioreactors that represent the different regions.

The dynamic organization and the ability to reorganize the volume and amplitude and sequence of zonal interconnections will allow the simulation of transient hydrodynamic phenomena that could occur in large-scale bioreactors. A primary application would be to use the system to optimize cellular phenotypes so that their production of desired biological products is tolerant of the breadth of conditions in the various zones. Another application would be to enable the serial sequencing of the steps in differentiating stem cells to a desired phenotype, by having the movement of effluent or cells from one microbioreactor to another represent natural cellular migration or spatial separation of differentiation steps, for example as occurs in the differentiation of various cells of the immune system during embryological development. The system could also be useful for the automated loading of different cell types into scaffolds or organ chips or other systems used for regenerative medicine that require the structured co-culture of multiple cell types, wherein a selected bioreactor contains one of the needed cell types.

Specifically, this invention relates to a network platform comprising a fluidic network comprising one or more pumps, and one or more valves; and a plurality of fluidic modules interconnected by the fluidic network of the one or more pumps and the one or more valves to allow controlled transfer of suspended cells and fluids from one fluidic module to another, or self-circulating.

In some embodiments, the network platform further comprises at least one input media reservoir and/or at least one collection reservoir in fluidic communication with the fluidic network for providing inputs and/or collecting outputs of any one of the plurality of fluidic modules, respectively.

In some embodiments, the plurality of fluidic modules comprises bioreactors, wells, organs-on-chips, chemostats, or a combination of them.

In some embodiments, each of the plurality of fluidic modules is individually perfusable. In some embodiments, a rate of perfusion of each fluidic module is controlled by at least one of the one or more pumps and the one or more valves.

In some embodiments, the one or more valves are a rotary planar valve system that is operably regulated with a single motor as a time-domain fluidic multiplexer to move samples between each and every one of the plurality of fluidic modules.

In some embodiments, the one or more valves comprise an N×M crossbar valve that operably connects the at least one input media reservoir to the inputs of any one of the plurality of fluidic modules, and the outputs of the plurality of bioreactors to either the input of one of the bioreactors or the at least one collection reservoir, wherein each of N and M is an integer greater than zero.

In some embodiments, the one or more valves comprise n two-state crossbar valves, thereby creating 2ⁿ possible valve states, wherein n is an integer greater than zero.

In some embodiments, the fluidic network is a continuously pumped fluidic network configured to ensure that living cells are in tubes for only very short intervals of time, by being moved directly from one bioreactor to another without intermediate storage.

In some embodiments, the one or more pumps and the one or more valves are configured to ensure that all fluid lines are promptly washed to avoid the trapping or storage of cells in sub-optimal environments.

In some embodiments, the fluidic network further comprises a separating means coupled with the one or more pumps and the one or more valves for separating cells such that certain cells are recirculated to one fluidic module while others are allowed to be moved to another.

In some embodiments, the separating means comprises a filter or other means to retain all cells within a fluidic module and only extract the fluid from one fluidic module for transfer to another.

In some embodiments, the separating means comprises a tangential flow filter, an alternating tangential flow filter, spiral cell separators, or other means.

In some embodiments, the fluidic network is a single large-scale crossbar valve system that operates with a single pneumatic, vertical via, rotary, or other mechanical valve at each intersection between every fluidic module inflow and outflow line, with a pump on each of either the inflow or outflow lines, or a dynamic multi-stage interconnection network that uses multiple smaller-scale crossbar or other valves.

In some embodiments, the fluidic network is a dynamically reconfigurable network.

In some embodiments, the interconnections of the plurality of fluidic modules are configured to create or simulate biological systems in which there are large-scale spatial gradients that support a variation in microbial composition.

In some embodiments, the interconnections of the plurality of fluidic modules are configured to allow any or all of the plurality of fluidic modules to connect to any other or all of the other fluidic modules.

In some embodiments, the plurality of fluidic modules is configured to serve as a physical but smaller scale model of the heterogeneous zonation within a larger reactor, and thereby to support the optimization of cell lines to ensure efficient bioproduction by cells upon scale-up.

In some embodiments, the plurality of fluidic modules comprises multiple microbioreactors that are linked together into a single combined bioreactor system to operably simulate the traversal of a cell through the different zonal conditions of the industrial bioreactor, creating a small-scale system that is able to model the conditions within an industrial-scale bioreactor.

In some embodiments, the plurality of fluidic modules comprises multiple microbioreactors configured to simulate spatiotemporal heterogeneities that are inherent in large-scale bioreactors, such that each microbioreactor represents a region or zone with a set of cell culture parameters including cell density, cell replication rate and division state, pH, shear stress, temperature mixing rates, and the concentration of nutrients, metabolites, oxygen, carbon dioxide, and other gases.

In some embodiments, the network platform is usable in combinatorial chemical processing, in which aliquots of different chemicals are combined or split.

In some embodiments, the network platform is usable in synthetic biology and/or DNA computing, in which aliquots of specifically coded RNA or DNA, or other molecular sequences are combined or separated.

These and other aspects of the invention are further described below. Without intent to limit the scope of the invention, exemplary instruments, apparatus, methods, and their related results according to the embodiments of the invention are given below. Note that titles or subtitles may be used in the examples for convenience of a reader, which in no way should limit the scope of the invention. Moreover, certain theories are proposed and disclosed herein; however, whether they are right or wrong, in no way should they limit the scope of the invention so long as the invention is practiced according to the invention without regard for any particular theory or scheme of action.

### Problem Solution

Two possible solutions are known to reduce the loss of productivity resulting from heterogeneous growth conditions in large bioreactors:
1. Change the design of the bioreactor to reduce the heterogeneity of key operational parameters; and
2. Engineer strains to be more resilient to the fluctuation of environmental conditions.

The difficulty with the first solution is that heterogeneity is inherent to large-scale bioreactors as a result, among other things, of the greater difficulty in stirring larger volumes. For this reason, it is believed that strain engineering provides a more robust solution to mitigate the losses in biochemical production.

To assess the enormous number of combinations possible in genetics and cellular biology, robot-led high-throughput experimental methods have emerged. These robotic methods reduce the amount of time, cost, and labor required to characterize and engineer microorganisms.

Here we describe how a previously reported Continuous Automated Perfusion Culture Analysis System (CAPCAS), which is disclosed in U.S. Pat. No. 11,447,734, which is incorporated herein by reference in its entirety, is not only capable of conducting massively parallelized, high-throughput strain characterization and media optimization experiments in milliliter bioreactors, but also recapitulating the effects of the predictable dynamics of cell and media transport between different bioreactor zones. This patent application describes how proper application of pumps and valves enable the adjustable configuration of small bioreactors for use in scale-down modeling of industrial bioreactors.

### Scale-Down Models

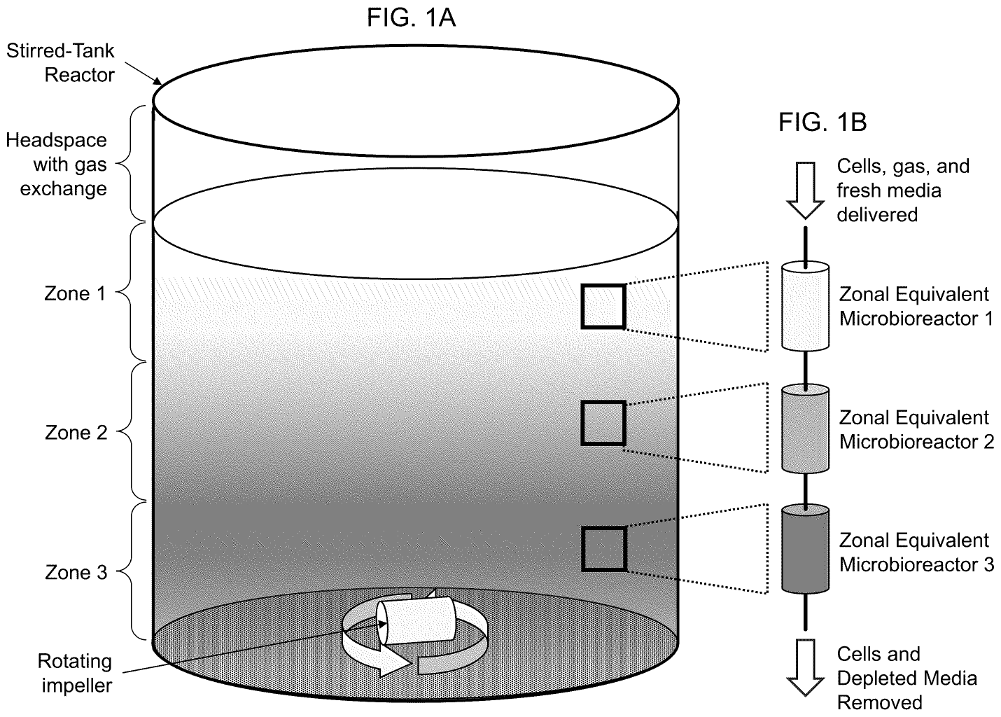

Scale-down model systems recapitulate the growth of cells under dynamical spatiotemporal conditions as are seen within a specific industrial bioreactor. Scale-down models are bioreactor systems composed of two or more small-scale bioreactors used to recreate some of the varying environmental conditions of a heterogeneous large-scale bioreactor. The transfer of cells through two or more heterogeneous compartments simulates the movement of cells through regions of varying conditions within an industrial reactor. FIG. 1A shows schematically the compartmentalization of the continuous distribution of conditions in a single large reactor. In this toy example, the diffusion of oxygen through three zones can be represented as three small bioreactors connected in series (FIG. 1B), with Zone 3 at the bottom of the tank most depleted of both oxygen and nutrients as compared to Zones 1 and 2 because of the greater distance from the oxygenating head space that can also absorb carbon dioxide from the media. To create a scale-down system, the standard approach is to use small reactors that replicate the conditions within the equivalence zones. Note that FIG. 1A shows a gradient of conditions with the region most depleted of nutrients being the bottom and the least depleted the top, while the three smaller bioreactors have three different sets of culture conditions.

Scale-down models of industrial reactors can be used to characterize the productivity of researched strains under industrial conditions without using industrial-scale bioreactors.

The mathematical principles of scale-down systems arise from the field of multi-scale modeling within computational physics. The molecular dynamics analogs to scale-down models of bioreactors are course-grained models of atomic systems. Course-graining is used to reduce the number of trajectories needed to be calculated in a system by approximating multiple atoms from an atomic system into a single bead. This reduces the number of bodies in the system and thus the number of computations. In course-graining, the potential energy produced by a neighborhood of atoms is approximated as a single bead that possesses the potential of the average potential energy of the atomic neighborhood. The mathematical foundations of this technique arise from Liouville's theorem, which states that a volume of phase space volume remains constant in the course of time, no matter where the point moves. This theorem essentially justifies the approximation of a system by a series of coarse representations of the actual phase-space conditions.

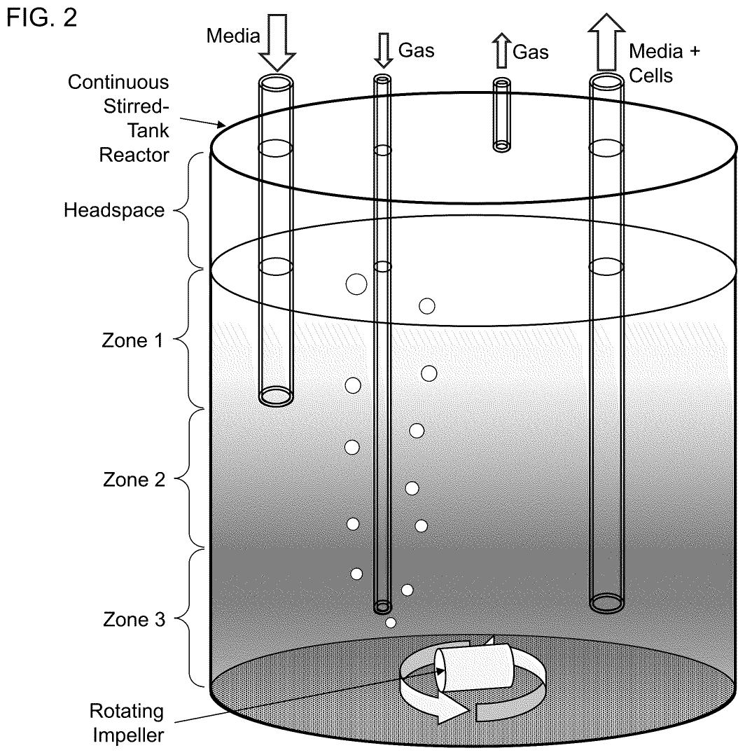

The overly simplified bioreactor in FIG. 1A illustrates the need for a zonal model. The stirred tank reactor (STR) in FIG. 2 is more realistic, in that it includes sparged oxygen delivery with a gas delivery tube, a gas vent tube, and a tube to deliver media and another to harvest media and cells from the reactor. The nature of the gradients in this bioreactor will be determined by multiple factors, including the distribution of bubble sizes, how gas exchange varies throughout the bioreactor, the fluid flow pattern and temperature differences throughout the bioreactor, the spatial distribution of delivered nutrients, how cell density affects the effective viscosity of the media-cell slurry, and whether the bioreactor is operated in batch mode, fed-batch mode, continuous feed with cellular recirculation, or as a chemostat without cellular recirculation. The recovery of cells from any effluent stream and their return to the reactor chamber will clearly affect the performance and efficiency of the bioreactor. That said, FIG. 2 still shows a simple vertical gradient, where in fact different bioreactor parameters will have differing spatial distributions. The larger the reactor, the larger and more diverse the gradients can be.

### Scale-Down Systems

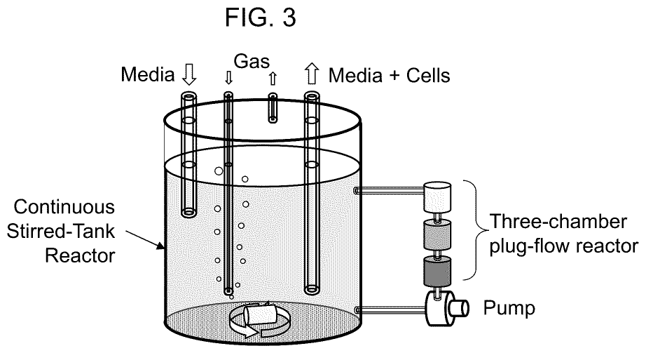

Many variations of scale-down systems have been designed, each possessing its own advantages and disadvantages. Although there are many variations, most are derived from the classical two-compartment scale-down system composed of a continuous stirred reactor (CSR) and a plug flow reactor (PFR) connected in series, as shown in FIG. 3. The two-compartment system directs subsets of the homogeneous CSR population into the heterogeneous, three-component PFR to induce stresses on the cells that mimic the depletion of oxygen and nutrients that occur when the cells traverse from the enriched to the depleted regions of the large-scale bioreactor. Note that the bioreactors in FIG. 3 are smaller than that in FIG. 2, and because stirring is more effective for small volumes, there is no gradient in the CSR in FIG. 2. Theoretically, the PFR moves fluid through the plug such that the cross-sectional area perpendicular to the length of the plug is composed of a homogeneous population. As this uniform population proceeds down the plug, the cells become more and more stressed due to depletion of chemicals used for their metabolism, such as oxygen or glucose. This two-compartment scale-down system works because we can consider the large-scale reactor as a discretized population consisting of low and high mixed regions, i.e., different zones of equivalence. These populations undergo different environmental conditions, such as high shear stress but uniform nutrient concentration for the high mixed region, and low shear but varied nutrient concentration for the low mixed region. Various modifications of the two-compartment system have been made to account for different desired characterization parameters and the benefits or detriments of aspects of the design. These modifications include adjusting the number of compartments, the volume of the CSR, aeration and feeding ports on the PFR, variations in reactor shape, impeller and gas sparger design, the placement of internal baffles, and many others.

Although these modifications have added to the capabilities of scale-down characterization systems, no instrument is known that is capable of creating a formulated distribution of stressed cells. A system like this would add to the generality of the scale-down system since any distribution of stressed cell populations could be programmatically and consistently generated for characterization. In addition, scale-down systems are typically used to hold a single parameter constant and characterize how the productivity of the system changes when the volume of growth media changes. However, holding a single variable parameter constant, such as shear stress or dissolved oxygen, neglects the covariance that occurs between the other variables that may result in unforeseen losses of productivity.

### Industrial Bioreactor Characterization

To model the heterogeneous conditions of industrial bioreactors, proper design of a scaled-down model must be performed. Great care must be taken to ensure that the results from the scaled-down system are applicable to the scaled-up system that is modeled. Due to the high costs of research and the need for the results to be representative of the physical system, considerable effort is often taken to understand the heterogeneities present in each unique industrial bioreactor. Generally, the starting point is to determine the operating variables whose fluctuations present the greatest risk to productivity loss. Once key operating variables are determined, the range of values of these variables in all regions of the industrial reactor must be estimated for the specific industrial bioreactor targeted for scale-down modeling. It is known that the heterogeneities in an industrial bioreactor are the result of at least three factors that differ for each biologic production process: the organism grown, its metabolic needs, and the physical bioreactor used to grow the organism. Ultimately, these heterogeneity factors are governed by how the biology of the organism interacts with the fluid properties and metabolic activities of the media-cell-metabolite slurry.

After the spatiotemporal heterogeneities of operational variables within the industrial-scale bioreactor have been determined, an in silico model, i.e., a digital twin, might be created that recapitulates the industrial bioreactor's behavior. However, proper characterization of an industrial bioreactor comes with great difficulty, however, given the lack of data surrounding their conditions and behavior, and the challenges of sampling a large reactor at a fine enough mesh of interior points without disturbing the flow fields or violating the Good Manufacturing Practice guidelines that regulate access to large reactors used for pharmaceutical production. There also exists great variability in the heterogeneities present from reactor to reactor, and within a reactor as a function of cell growth and time, further adding to the challenge of reactor characterization.

Given these difficulties surrounding characterization of empirical industrial bioreactors, computational techniques have become pivotal to understanding bioreactor characteristics. One approach that is used for characterizing the fluid properties of a bioreactor without explicit measurements of the system is computational fluid dynamics (CFD). Very generally, CFD involves integrating the Navier-Stokes partial differential equation, in its most general, convective form,

$$ρ ( \frac{\partial u}{\partial t} + u \cdot ∇ u ) = - ∇ p + ∇ \cdot \{ μ ( ∇ u + {( ∇ u )}^{T} - \frac{2}{3} ( ∇ \cdot u ) I ) + ζ ( ∇ \cdot u ) I \} + ρ g , \qquad ( eq . 1 )$$

where ∇ is the tensor gradient, ∇• is the divergence, I is the identity tensor, ζ is the volume (or second) viscosity, μ is the dynamic viscosity, ρ is the density, u is the flow velocity, ρ is the pressure, t is time, r, and g represents body accelerations acting on the continuum, for example, gravity, inertial accelerations, electrostatic accelerations, etc. Each of these variables can have significantly different values throughout the bioreactor volume.

To integrate the Navier-Stokes equation, the volume of the bioreactor must be discretized into smaller volumes, called a mesh, enabling integration that is made possible by the imposition of boundary conditions on the equation. The result of a conventional CFD simulation is a vector field describing the flow velocity at each point of the discretized bioreactor. Many variations of CFD simulations have been developed to recreate different fluid phenomena, such as shear stresses from aeration bubbles, shearing resulting from impeller turbulence, differences in viscosity and density associated with the injection of nutrients, and many others. There is a plethora of variations of CFD, but ultimately the specific CFD method used should be chosen based on the key operational variables that are going to be modeled with the scale-down system.

At the cellular level, the biology of the organism can be modeled with any of several techniques, such as flux balance analysis (FBA). The fundamental assumption of FBA is that the system is at steady state with no change in concentration such that

S·v=0, (eq. 2)

where S is the stoichiometric matrix of metabolic reactions and v is the vector of metabolic fluxes. In recognition that the population of cells being studied may itself be heterogeneous, this expression can be coupled with population balance equation (PBE)

d/dt∫_{Ω}_{ₓ}₍ₜ₎dVₓ∫_{Ω}_{₂}₍ₜ₎dVᵣƒ(x,r,t)=∫_{Ω}_{ₓ}₍ₜ₎dVₓ∫_{Ω}_{ᵣ}₍ₜ₎dVᵣh(x,r,Y,t), (eq. 3)

where h(x, r, Y, t) denotes the birth rate of particles per unit volume of particle state space, (x, r) is the particle state vector denoting the average number of particles with particle properties, and f(x, r, t) is the continuous phase in which the particles are dispersed. FBA operates under the assumption of the law of mass balance, which states that the total mass of the system should remain constant. Solving eq. 2 for the flux vector results in a description of the dynamics of the metabolism of the organism. With an extension to FBA, dynamic FBA (dFBA), a temporal description of the concentration of nutrients taken in and waste excreted out of the cell can be determined. The PBE is an integro-partial differential equation that gives the mean-field behavior of a population over time.

To create a robust model capable of simulating the complex phenomena of an industrial bioreactor, a computational framework that simulates both the fluid and biological properties has been developed. This bioprocess characterization framework utilizes a coupled dFBA, PBE, and CFD model and is able to simulate the effects of both the biological behavior of the microbial population and the fluid dynamics of the slurry within a bioreactor.

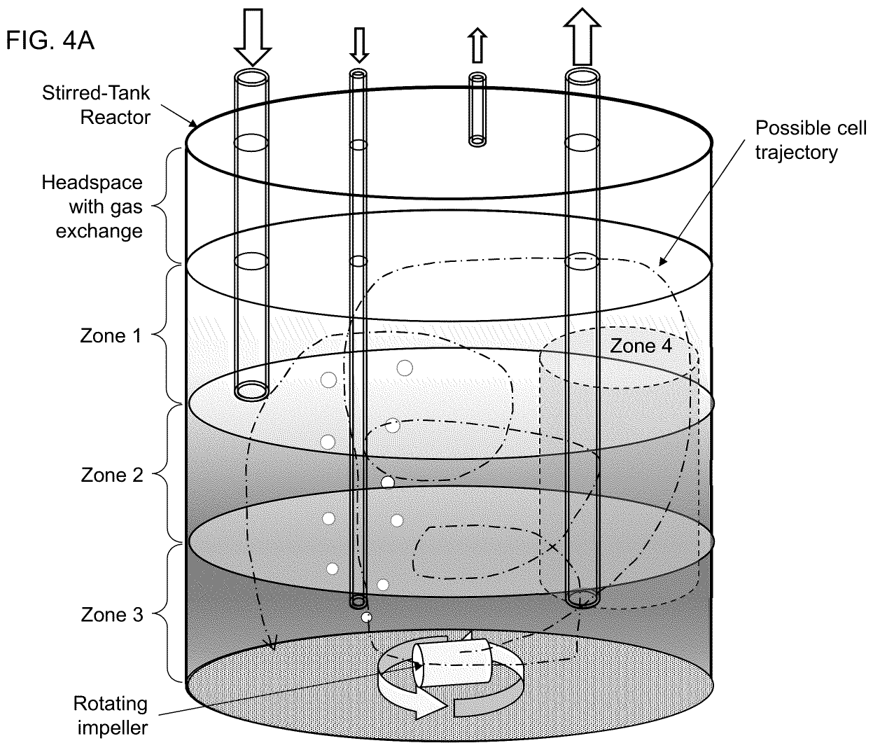

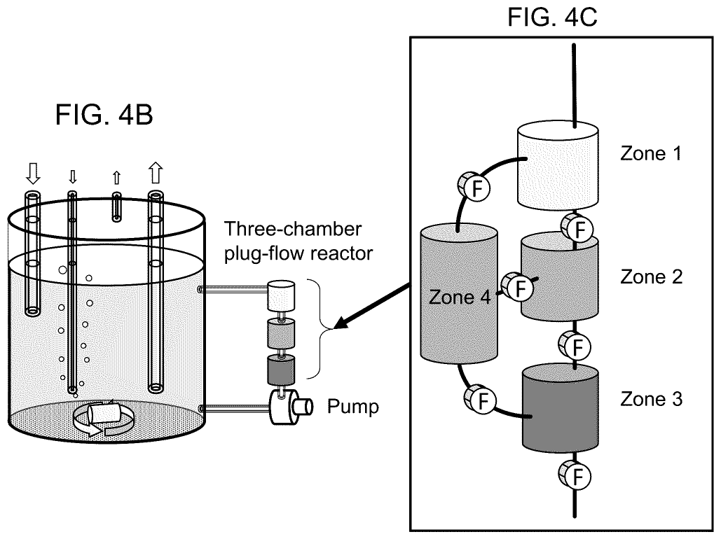

As shown in FIG. 4A, the continuous conditions of the bioreactor can be discretized into zones of relative conditional equivalence, shown as our original Zones 1-3 and an additional Zone 4. A single zone would normally contain a very fine computational mesh that contains a very large number of small elements. After a CFD simulation of the hydrodynamics within the tank has been performed, a vector field can be created describing the motion of liquid within the bioreactor. Using integration methods such as Euler's method, a specific path for a cell can be estimated, as shown by the one-of-many possible cell trajectory in FIG. 4A. In practice, the zones describing a real bioreactor would be larger in number and more complex in shape than shown in FIG. 4A.

It is beyond the scope of this disclosure to recapitulate all steps in the process of parameterizing, specifying, and coding such a zonal model, but the key initial one is to determine as many physical and biochemical parameters as possible for as many locations as is feasible within the bioreactor tank. Typically, the need for a model arises from the impossibility of obtaining sufficient data to describe completely the spatial variations of cell culture conditions throughout the volume of the tank and, more important, the challenges in determining the statistical distribution of the possible cellular trajectories and residence times in each zone of the tank. The mass transport of both liquid and gas can be simulated using CFD at a chosen spatial resolution, i.e., model mesh size. Criteria can be set that allow the identification of various zones, and FBA, dFBA, and PBE analyses conducted for each zone. The range of trajectories, such as the single one suggested in FIG. 4A, leads to a distribution in the exposure time of cells to each of the different zonal conditions, which in turn can lead to significant changes in the cellular phenotypes within the tank and a concomitant reduction of cellular productivity. Because the gradients that are common in large tank bioreactors are difficult to recapitulate in small tank bioreactors, one or more plug-flow modules can be added in series or in parallel, as shown in FIGS. 4B and 4C. The specific flux through each component can be regulated with an active or passive flow control element, F, between each zone. A challenge is to properly specify both the topology of the network and the strengths of the couplings Fᵢⱼ between reactor i and reactor j.

### Zonal Equivalence

Once the industrial bioreactor has been characterized via computational techniques, the creation of a physical scaled-down system can proceed. The parameter used to distinguish between CSR and PFR flow behavior is the Bodenstein number, B₀.

$${B}_{0} = \frac{u \cdot L}{{D}_{ax}} , \qquad ( eq . 4 )$$

where u is the flow velocity, L is the length of the reactor, and Dₐₓ is the axial dispersion coefficient. The Bodenstein number describes the ratio of the amount of substance introduced by convection to that introduced by diffusion.

If the Bodenstein number is greater than 10, the tank is considered a plug flow reactor (PFR); if it less than 10, the reactor is considered a continuous stirred reactor (CSR). Scaled-down systems model heterogeneous variables within industrial bioreactors by discretizing the effectively continuous distribution of growth conditions within the reactor into zones of approximately conditional equivalence. The zones are then subdivided into smaller discrete elements. The conditions within each discretized zone within the industrial reactor are then replicated using small laboratory bioreactors (FIGS. 3 and 4C). Linking several small-scale bioreactors together into a single combined bioreactor system simulates the traversal of a cell through the different zonal conditions of the industrial bioreactor, creating a small-scale system that collectively is able to model the conditions within an industrial-scale bioreactor. Within each small bioreactor of the small-scale system, the concentration of nutrients, oxygen, waste products, pH, shear rate, Bodenstein number, and various other conditional variables can be controlled to produce the behavior desired. Of course, different biological systems possess different sensitivities and critical operational parameters that must be scaled-up.

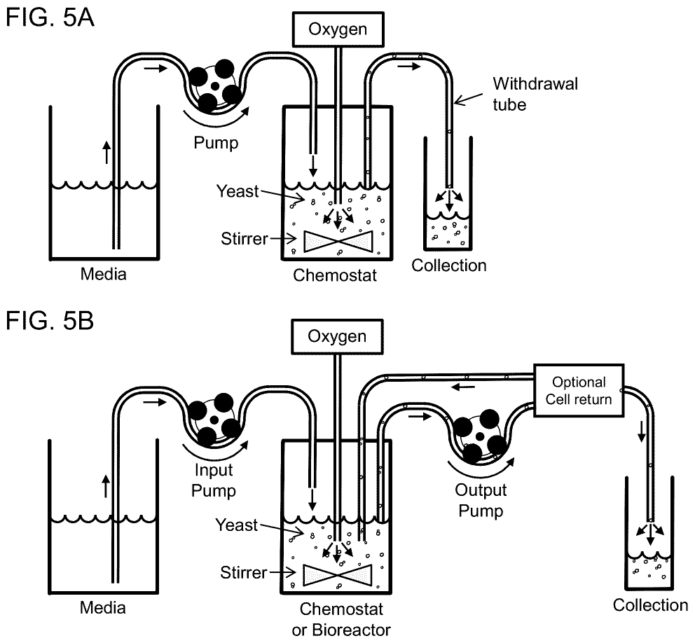

It is useful to examine the simple perfused bioreactors shown in FIGS. 5A and 5B, which show chemostats or perfusion bioreactors containing microbes such as yeast, with a pumped media source, stirring, oxygen delivery and exchange of carbon dioxide, and either passive (FIG. 5A) or active (FIG. 5B) removal of the excess media. In FIG. 5A, the reactor operates as a chemostat with fixed internal volume, with a pump delivering media at a controlled rate and a passive withdrawal tube whose depth of penetration into the tank sets the volume of media within the reactor. The yeast density within the reactor will remain constant as long as the media delivery rate is matched by the yeast growth rate.

With the configuration of the chemostat and pumps shown in FIG. 5B, it is possible to include a separation means that returns all or a fraction of the cells in the effluent stream back to the original chemostat prior to the effluent being delivered to a reservoir or connected to one of the crossbar or other networks described below. This can be done with tangential flow filtering, alternating tangential flow filtering, spiral cell separators, or other means. This allows all or at least the majority of cells to remain captured in each bioreactor, and the media and whatever metabolites or extracellular vesicles it contains to be exchanged for that in other bioreactors, as might be useful in the controlled differentiation of stem cells or other progenitor cells such as multipotent stromal cells (MSCs). The consumption or secretion of media factors in one zone is then reflected in the media delivered to an adjacent zone. In contrast to FIG. 5A, FIG. 5B has an active output pump that can, if desired, be used to power such a tangential flow filter, an alternating transverse filter, or a spiral cell separator that is designed to return cells to the bioreactor but remove excess media from the bioreactor and deliver it to another reservoir or bioreactor.

The proper specification of the interconnections between the various bioreactors shown in FIGS. 3, 4B and 4C is critical as to whether this small-scale system will adequately recapitulate the effects that zonation in the large reactor in FIG. 4A will have on cellular phenotype and productivity, and the extent to which a CFD/FBA/dFBA/PBE model can successfully use small reactor data to predict large reactor performance. This invention addresses the challenges associated with regulating the flow rate and retention time of media in each one of multiple, interconnected small bioreactors.

### System Configuration (Bidirectional Microformulator)

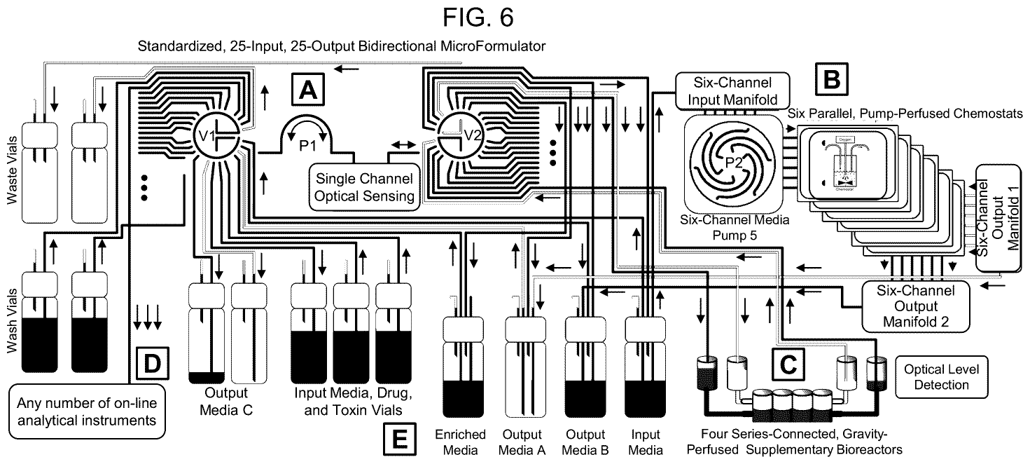

As one means to enable the modifiable transfer of liquid from one bioreactor to another, the bidirectional microformulator shown in FIG. 6 can be utilized such that it will move fluids under time-division multiplexing. The original implementation of the microformulator was unidirectional, in that media was formulated by collecting aliquots of different media components and mixing them in the desired proportions. The application was unidirectional, in that the microfluidic pump between the input selector valve and the output director valve operated in only one direction—from the input reservoirs with media components to the output reservoirs with the individually selected component mixtures. In contrast, the bidirectional microformulator is designed to mix media from multiple chemostats and series-connected, gravity-perfused supplementary bioreactors, with the microfluidic pump running in both directions so as to fill a particular upstream input reservoir with material extracted from a down-stream bioreactor. In this mode, a subset of what were previously the input reservoirs is reassigned a role as temporary storage reservoirs, or buffers, for later direction to one or more other bioreactors. In FIG. 6, the modules are interconnected and operated as follows. A) The bidirectional microformulator with input selector valve V1, pump P1, and output director valve V2. Because the pump can operate in either direction and the input and output valves are connected to reservoirs or bioreactors, fluids can be withdrawn from any reservoir(s) or bioreator or added to any other reservoir(s) or bioreactors. B) The six parallel-perfused chemostats or bioreactors are perfused by the microformulator. C) The four series-connected, gravity-perfused supplementary bioreactors can be connected in series with one or more pump perfused chemostats by directing the media to flow through an intermediate reservoir and the bidirectional microformulator. D) The connection to one or more analytical instruments, generally by use of a selector valve, a cut-in-valve, or a sensor valve allows quantification of the state of the cells and effluent being produced. These valves can service multiple microformulator lines and multiple analytical instruments. E) A plurality of reservoirs store input, intermediate, and output solutions. This approach is centered on the time-division multiplexing of the microformulator in (A) and the transient storage of bioreactor effluent in (E) and its subsequent redirection to another bioreactor.

While time-division multiplexing provides great flexibility in moving, storing, and combining a large number of different media components, it is intrinsically a repeated, small-batch process. When the media components being retrieved from another bioreactor contain living cells, this system has the disadvantage of having metabolically active cells residing in tubing and reservoirs without proper levels of nutrients and oxygen and prompt dilution and/or removal of waste products. This invention addresses this problem by using continuously pumped fluidic networks to ensure that living cells are in tubes for only very short intervals of time, and are moved directly from one bioreactor to another without intermediate storage. The proper design of the pumps and valves and their operational protocols can ensure that all fluid lines are promptly washed after passaging cells to avoid the trapping or storage of cells in sub-optimal environments.

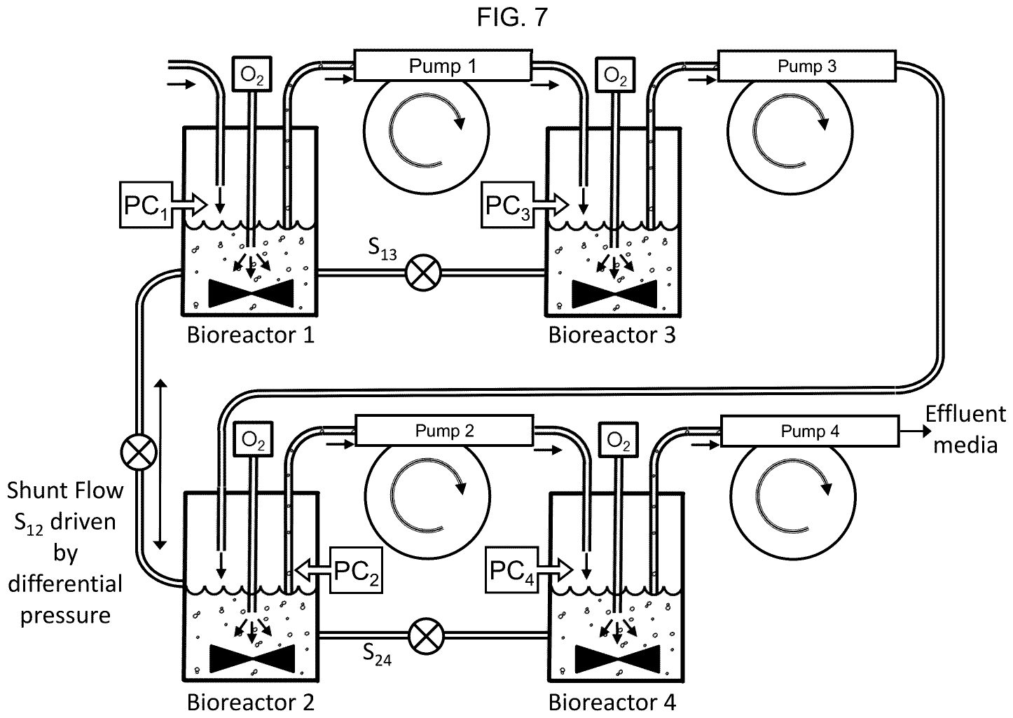

As an example of this approach, FIG. 7 shows four bioreactors permanently configured such that the effluent from Bioreactor 1 is passed to Bioreactor 3, whose effluent is pumped to Bioreactor 2. The effluent from Bioreactor 2 is then pumped into Bioreactor 4. The effluent media removed from Bioreactor 4 by Pump 4 is then the final output of the system of coupled bioreactors. In this embodiment, each bioreactor has independent oxygen delivery (O₂) and hence gas exchange, and uses a pressure controller (PC) and possibly a valved, external source of pressurized gas and a vent valve to regulate dynamically and under computer control the partial pressures of oxygen, carbon dioxide, and other gases in each bioreactor and hence the differential pressure between the four bioreactors. A regulated shunt flow (S) between pairs of reactors provides a non-pumped means to use pressure differentials to move fluid and possibly cells from one reactor to another with minimal shear forces. Valves connected to reservoirs containing wash solutions, not shown, could be used to flush lines were the fluid delivery between two bioreactors to be paused for any reason, such as to allow cells to divide in a batch mode prior to being transferred to another reactor.

FIG. 8A shows a generalized schematic that allows all possible connections between four bioreactors (BR), hence creating a completely connected network of bioreactors. Each bioreactor has independent media input (In) and output (Out), pressure regulation (PR), mixing, and temperature control. FIG. 8B shows a breadth of possible connection fluid-transfer elements (Fᵢⱼ) between Bioreactor i and Bioreactor j, including an open tube whose media transfer is regulated only by pressure differences between the two reactors, a pump that can move fluid in either direction, a valve to regulate pressure-driven flow, a check valve that ensures that pressure-driven flow can be in only one direction, or a cell filter (for example, a tangential flow filter, an alternating transverse flow filter, or a spiral separator, with associated pumps and cell return lines not shown). Other fluidic devices could be represented by Fᵢⱼ, and any Fᵢⱼ might represent series and/or parallel connections of multiple fluidic elements. Hereafter, FIG. 8A will represent the most general possible network connection scheme, with each bioreactor being connected to every other bioreactor by a permanent connection, each of which contains one or more of the possible Fij implementations in FIG. 8B.

While a single set of pressures could not cause the media to recirculate repeatedly though the four coupled reactors shown, in FIG. 8A, the closing of three valves between any bioreactor and its three neighbors could be used to pressurize that one reactor to subsequently, with the opening of one or more adjoining valves, move fluid into an adjacent one, two, or three reactors.

At any point, all the valves in the system could be transiently closed and the pressure in all bioreactors could be returned to baseline, all without any fluid movement. Hence the sequence to move fluid from one bioreactor to any other bioreactor would comprise closing all valves on one reactor, pressurizing that reactor, opening a single connecting valve to an adjacent one, closing all valves, depressurizing the system, and repeating the process for the next bioreactor along the intended pathway. Four such sequences could drive a bolus of media around the circuit in FIG. 8, without the use of pumps. Other sequences could thereby moving fluid over arbitrary defined pathways throughout the network, all without the need for a pump. Any sequence or set of sequences could be repeated ad infinitum to move fluid from any bioreactor to another.

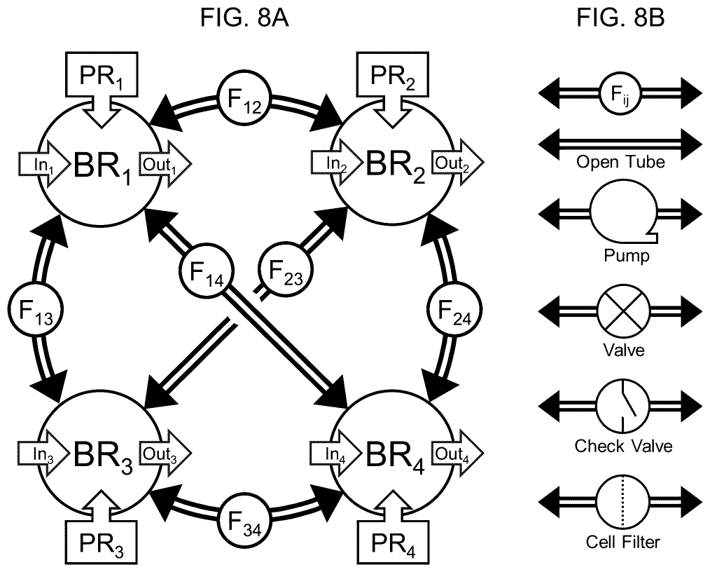

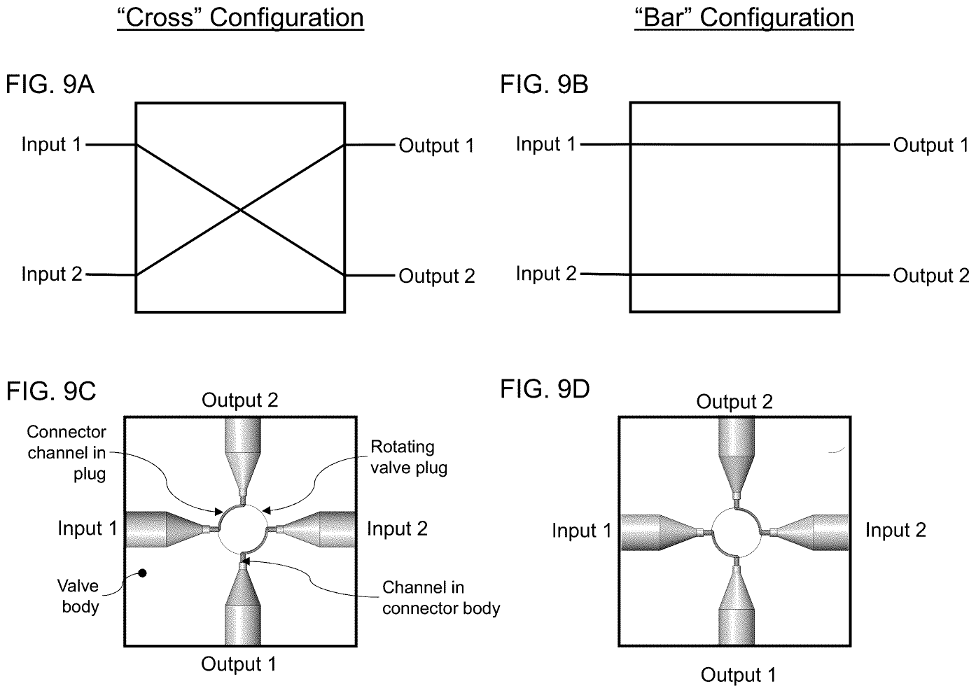

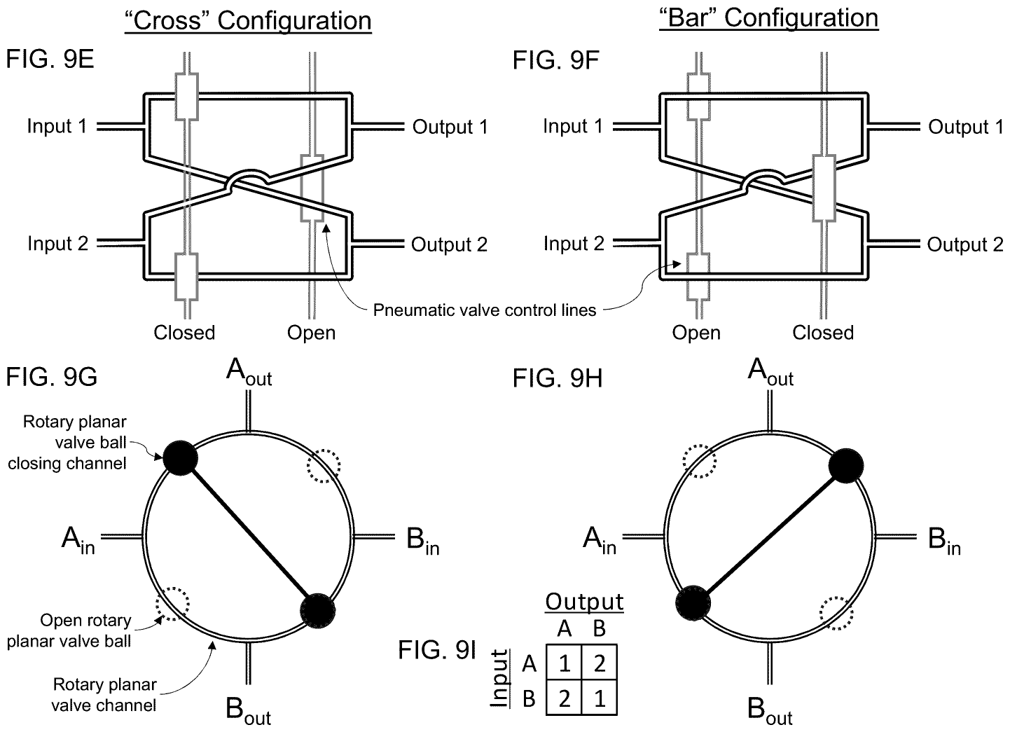

While the connections in FIG. 8B were shown as being permanent, an alternative approach is to use valved connections to create a dynamically reconfigurable network. FIGS. 9A-9I show several implementations of a four-port, two-by-two crossbar switch or valve that can be used to create more complicated fluidic networks. FIG. 9A and FIG. 9B show a generic electrical or fluidic crossbar in the “cross” and “bar” configurations, respectively. In an electronic crossbar, the box contains transistors or other switchable elements to make and break the appropriate connections. In a fluidic crossbar, the box contains fluidic valves implemented with any of several designs.

FIG. 9C and FIG. 9D show a two-position, commercially available rotary fluidic valve, where the rotating valve plug contains a pair of channels that connects Input 1 to either Output 1 or Output 2, while simultaneously connecting Input 2 to either Output 2 or Output 1. Rotating the valve plug by 90 degrees makes and breaks the four connections to switch from Cross to Bar configurations. FIG. 9E and FIG. 9F show a functionally equivalent pneumatic microfluidic crossbar valve, where the pressurization of one of the two valve control lines will seal either the Bar or Cross connections by occlusion of the channel beneath the pressurized control line. FIG. 9G and FIG. 9H show how a rotary planar valve (RPV), which we previously described in detail in U.S. Pat. Nos. 9,618,129, 11,135,582, and 11,465,144, each of which is incorporated herein by reference in its entirety, can be used to implement a crossbar valve by a 90° rotation of the valve actuator, which causes four caged balls to either open or close the adjacent fluidic channel. FIG. 9I shows the truth table for this valve, and the other 2×2 crossbar valves.

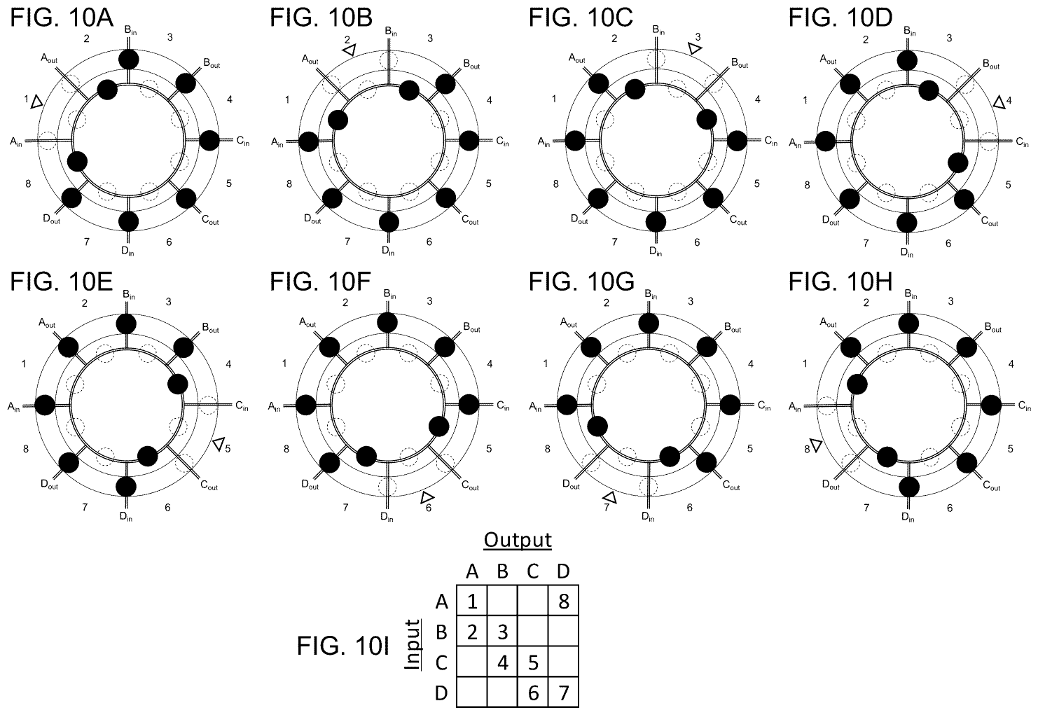

FIGS. 10A-10I show the eight states of a 4×4 rotary planar crossbar valve. Note that this is a blocking valve, in that a single input can be connected to two of the outputs, and other connections are not possible. With a rotary planar valve system, a 2×2 or a 4×4 or larger fluidic crossbar valve can be regulated with a one or more motors and can utilize time-division multiplexing to move media between each and every one of multiple chemostats or bioreactors.

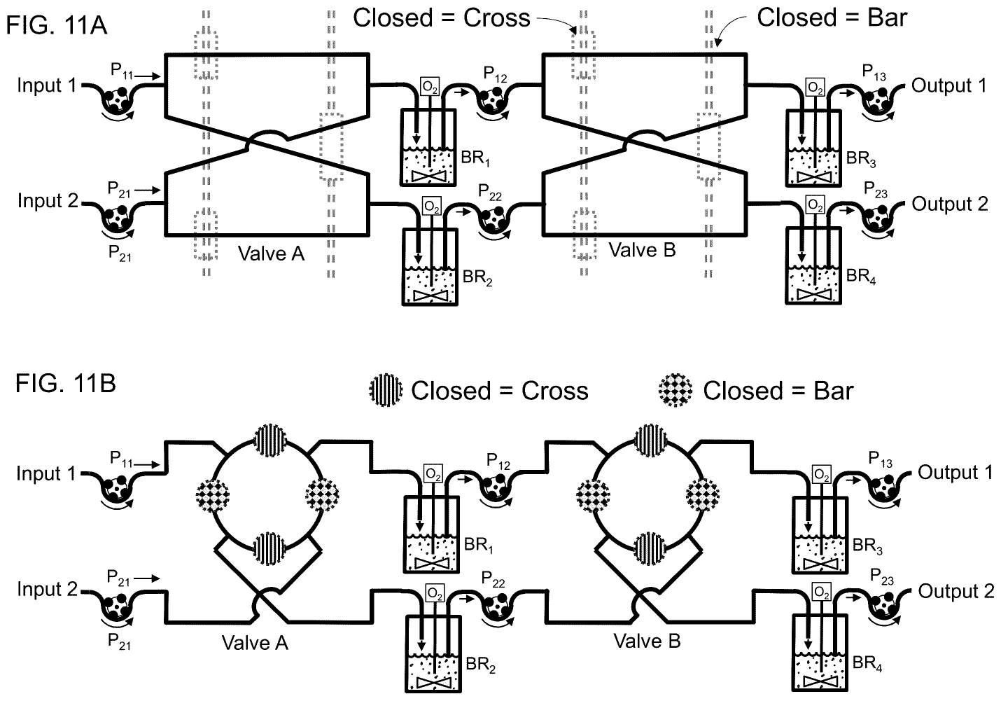

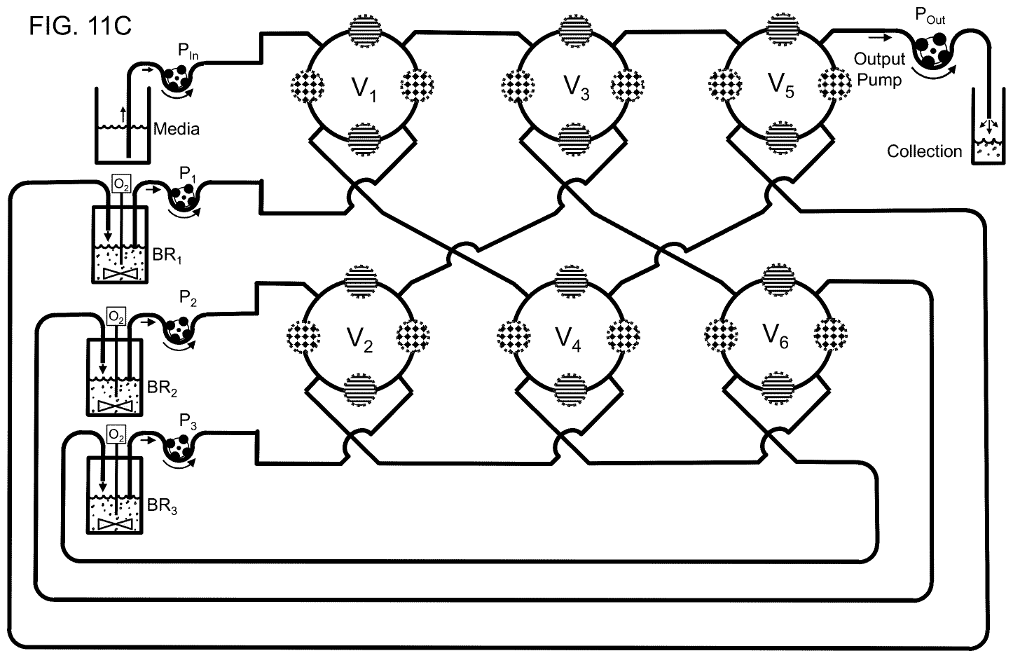

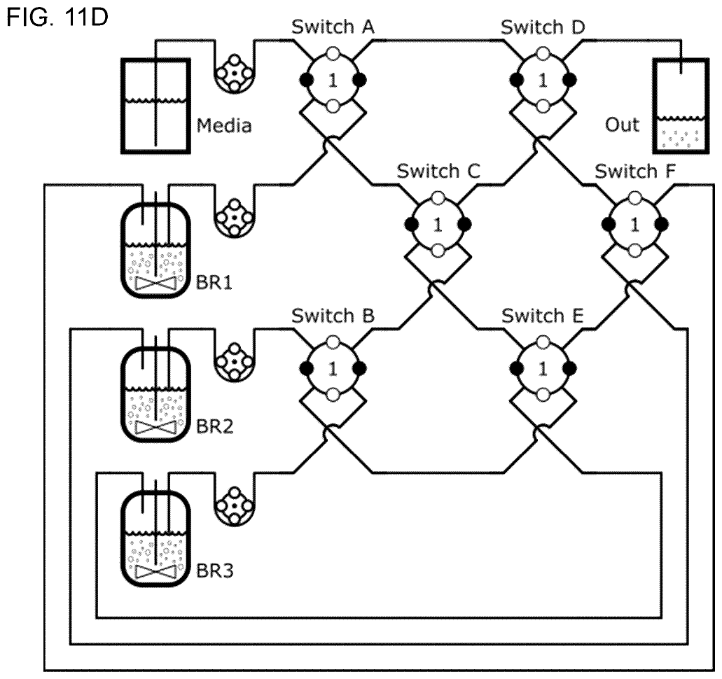

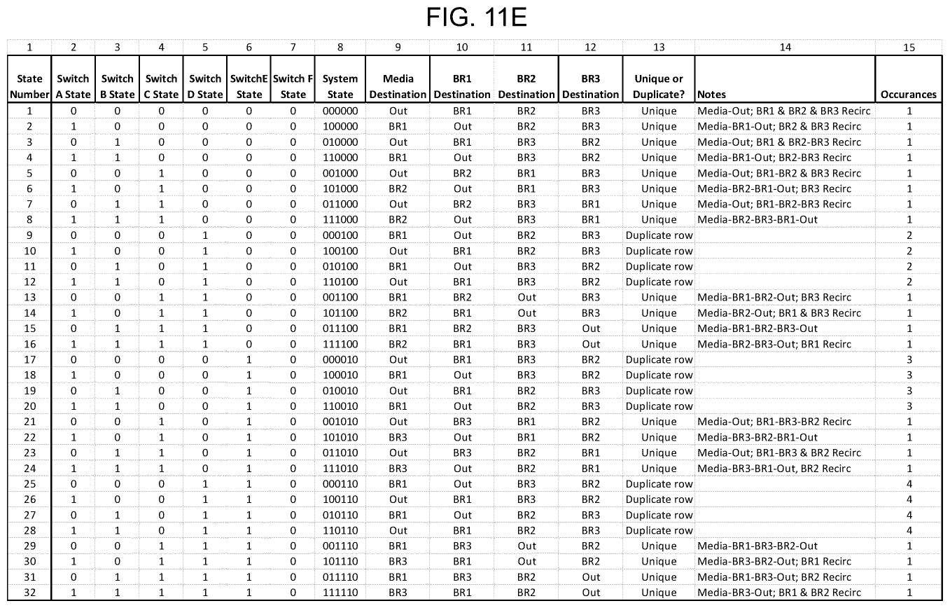

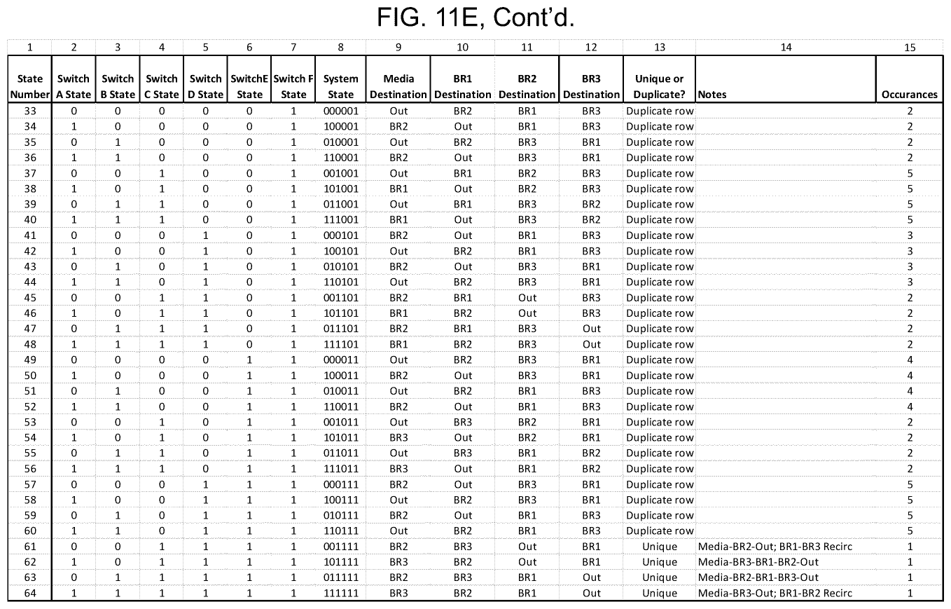

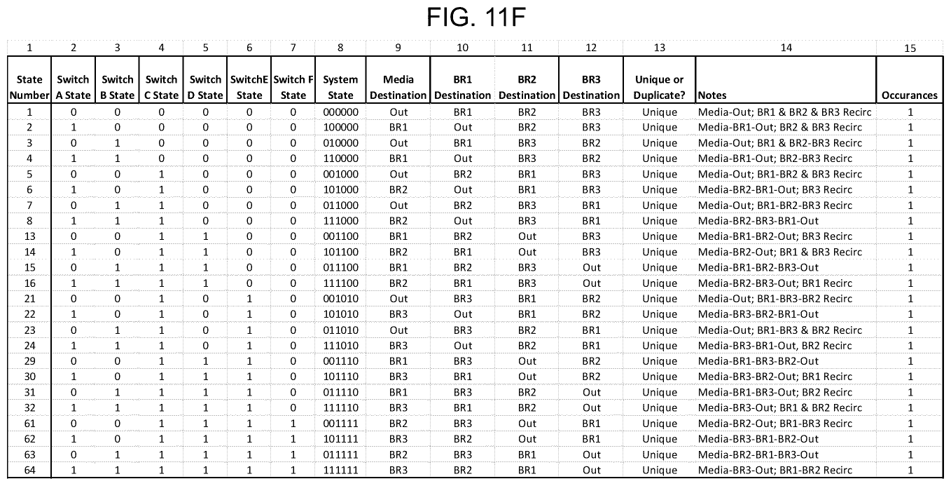

FIG. 11A and FIG. 11B show how a pair of 2×2 pneumatic or rotary planar crossbar switches can be connected in series with four bioreactors (BRs), each with media either delivered or withdrawn by an in-line pump (P). Crossbar Valve A determines whether BR₁ is perfused by Input 1 or Input 2 and BR₂ perfused by Input 2 or Input 1, respectively. Crossbar Valve B determines whether BR₃ is perfused by the output of BR₁ or BR₂ and whether BR 4 is perfused by the output of BR₂ or BR₁, respectively. Note that the state of the pneumatic valves can be maintained only if the pressure that is applied to either the Cross or Bar pneumatic control line is also maintained, i.e., pneumatic valves are not latching but require a continuous control signal. In contrast the rotary microfluidic valves in FIG. 11B only require transient application of power to the motor or actuator that rotates the valve plug or actuator by 90°. In practice, a single digital instruction bus driving an array of microprocessor-controlled motors will be more compact than a valve that requires not only fluidic lines but also pneumatic control lines.

FIG. 11C and FIG. 11D illustrate how six 2×2 rotary planar crossbar valves can be used to create a non-blocking 4×4 rotary planar crossbar valve that connects a single input media reservoir to the inputs of any one of three bioreactors, and the outputs of the three bioreactors to either the input of one of the bioreactors or the collection reservoir, labeled “Out”. Because the network is non-blocking, it can support four independent paths of connections across the network. Given that there are six two-state crossbar valves, there are 2⁶=64 possible valve combinations. FIG. 11E presents the truth table for the 4×4 rotary planar crossbar valve in FIG. 11D, showing a wide variety of configurations achievable by simply toggling one or more of the valves. Given that these valves can be switched quickly, it would be straightforward to use time-division multiplexing to rearrange on demand or periodically the connections between each of the bioreactors, the media supply, and the output. Such a network, for example, could also be used in combinatorial chemical processing, or even DNA computing, in which aliquots of specifically coded DNA sequences are combined or separated.

As shown in FIG. 11F, there are 24 unique possible connection configurations. Of the non-duplicates, several of these patterns are worthy of note:
There were six configurations where Media was delivered to one reactor, with the second and third in series and the last connected to Out, with all permutations of their perfusion order: 8 (111000), 15 (011100), 22 (101010), 29 (001110), 62 (101111), 63 (011111), and 62 (101111). These states are the best for long-term operation of the system, in that each bioreactor is continuously perfused.
Six reactors had Media delivered to two reactors in series, with the second being connected to Out, and a third self-recirculating: 6 (101000), 13 (001100), 16 (001100), 24 (111100), 24 (111010), 30 (101110) and 31 (011110). The self-recirculating reactor has neither media addition or withdrawal, which might represent a transient state of cells in a particular zone of a large bioreactor.
Three reactors had a single reactor connected to Media and Out, and the other two reactors self-recirculating: 2 (100000), 14 (101100), and 32 (111110).
Three reactors had one reactor connected to Media and Out, while the other two recirculated as a pair: 4 (110000), 61 (001111), and 64 (111111).
There were 6 configurations with “Out” in Column 9, indicating that input media was being sent directly to the output port without connecting to any of the bioreactors, but with the three reactors can be connected in different ways: with all three self-recirculating (1-000000); three circulating in series in one or the other direction (7-011000 and 21-001010); or one self-recirculating and the other two recirculating as a pair (3-010000, 5-001000, and 23-011010). Again, these states are best viewed as transient, batch phase, because none of the reactors have media being added or removed.

One of the great merits of this invention is that it allows rapid switching between various pre-selected states, thereby allowing the effects of zonation to be simulated dynamically by switching. Larger networks with more than three bioreactors would be able to simulate the interactions of a larger number of zones. A digital twin of this system would enable ready comparison of a living biological model with an in silico equivalent.

Note that because the valves shown in this embodiment are binary, i.e., open or closed, the valves are either open or closed, and there can be no proportional mixing. Because there are no branches in any of the lines, it is not possible with this network to connect two reactors in parallel. Such an arrangement would be possible with additional lines and switches to create branching elements that would allow the splitting or combination of two flows.

Originally developed for the fields of telephone exchange systems and computer networking, interconnection networks are modular networks that connect any input in the network to any output in the network. There are many networking topologies that are well understood in their relevant fields, and some can be applied within the scope of reactor vessel modeling, but the use of multiple small, interconnected bioreactors to recapitulate zonation in large bioreactors requires a potentially vast variety of reactor vessel configurations and the ability to change and mutate network topologies in a dynamic manner and create new topologies to represent reactor vessel parameters that either change smoothly with time or may even be chaotic. Parameters like reactor shape, impeller design and stir rate, fluid viscosity, gas injection, and temperature could alter dynamically the way nodes need to be connected. It is easy enough to change these topologies in silico, but the validation at the in vitro level must be mutable in a similar way and is currently tedious and error prone when performed with traditional laboratory equipment, hence the present invention.

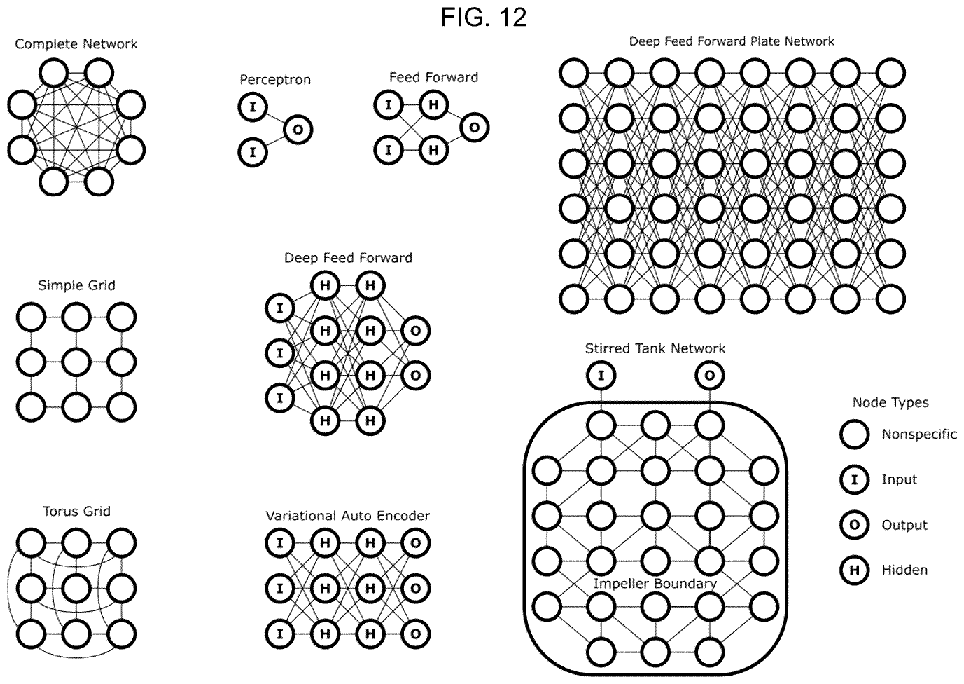

Network topologies like the Complete Network in FIG. 8 and FIG. 12 have the advantage that any node can communicate with any other node allowing multiple path topologies, but as the number of nodes increases, the connection complexity increases rapidly and they are not well optimized for multi-path parallel recirculating information. Simple network models like the fixed connections in FIGS. 7 and 11 and the Simple Grid in FIG. 12 are too rigid and do not allow communication from any node to any other node. This may better represent the physical space limitations of the reactor vessel but can be inefficient when used for recirculating models which best represent the cyclic nature of the vessels. The Toroid grid improves this capability but still suffers from similar inefficiencies and rigidity of node configurations. Implementing dynamic switching topologies like the crossbar and Bene networks between node layers offers similar any-to-any communication. Neural nets can model sequential communications between nodes and can include recurrent nodes and feedback nodes.

There are multiple variations of interconnection networks that have different blocking and nonblocking properties. FIG. 12 shows a number of network topologies that can be implemented in microfluidics and are relevant to this problem, summarized as follows:
Complete Network—Every node has a connection to every other node. Nodes have no unique functionality from each other.
Grid—Nodes are connected only to the nearest neighbor nodes in a specific grid pattern. Some nodes must communicate through other nodes to get information (fluid) from one side of the network to the other.
Torus—Nodes are connected only to the nearest neighbor nodes in a specific grid pattern, but edge nodes are directly connected to opposing edge nodes on each grid line. This lends itself to better modeling of continuously recirculating information.
Perceptron—Nodes have specific functions and are connected in a specific pattern where input nodes only talk to output nodes.
Feed Forward—Nodes have specific functions and are connected in a specific pattern and there are “Hidden” nodes between inputs and outputs but no interlayer connections.
Deep Feed Forward—Nodes have specific functions and are connected in a specific pattern and there are multiple layers of “Hidden” nodes with additional complexity between inputs and outputs. There are no interlayer connections and the number of nodes in the “Hidden” layers are often greater than the inputs or outputs and the number of output nodes is often n−1 of the input nodes.
Variational Auto Encoder—Nodes have specific functions and are connected in a specific pattern and there are multiple layers of “Hidden” nodes with additional complexity between inputs and outputs. There are multiple layers of “Hidden” nodes, but the layer widths are the same throughout.
Deep Feed Forward Plate Network—Nodes are nonspecific and utilize bidirectional connections which allow simulation of 3D continuously recirculating networks.
Stirred Tank Network—Nodes are nonspecific and utilize bidirectional connections which allow simulation of 3D continuously recirculating networks. Dynamic switching allows virtual features such as physical boundary layers within the virtual tank, for example the boundary between zones on opposite sides of the tank that cannot be in direct communication because of the flow patterns emanating from the impeller.

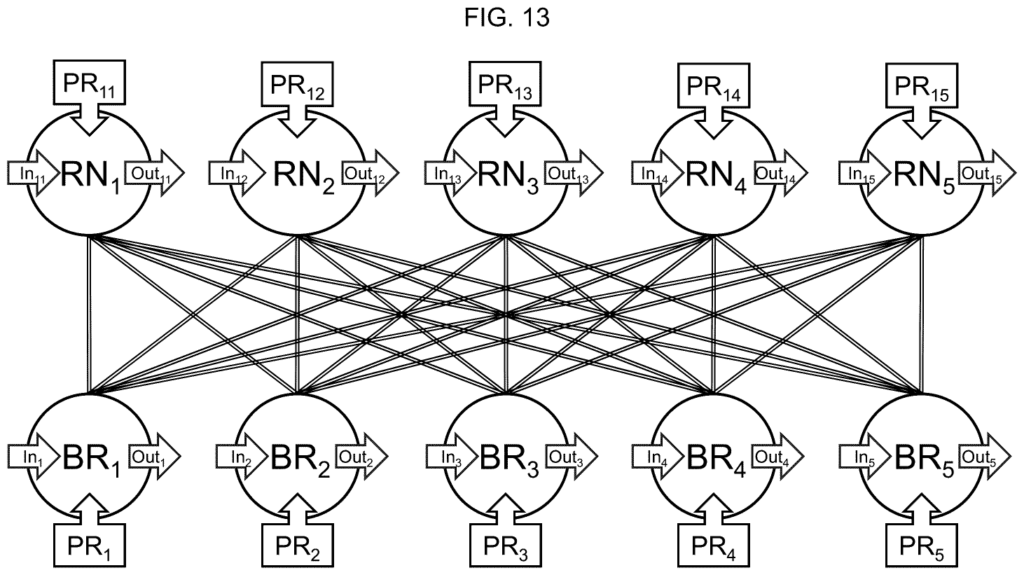

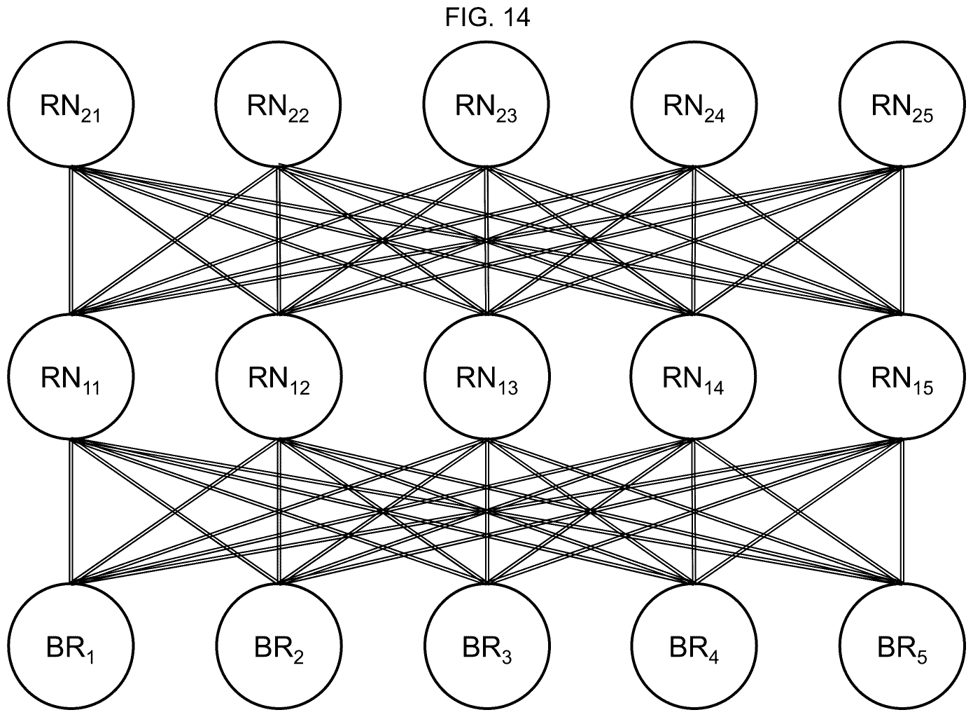

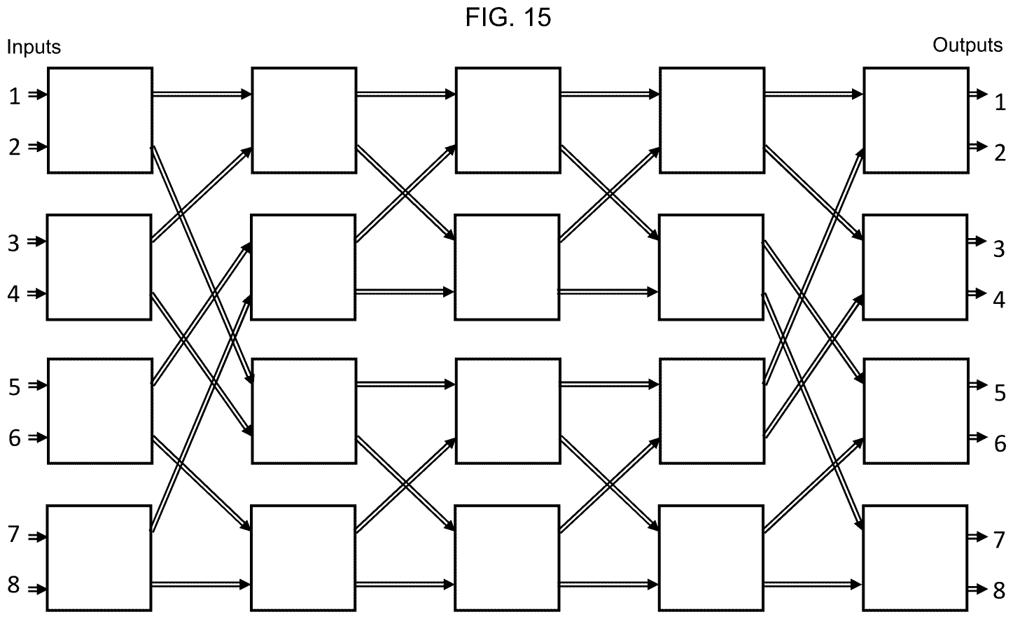

FIG. 13 and FIG. 14 show hypothetical networks where the five zones of interest in the bottom row of each figure (BR₁ through BR₅) are connected not by simple pipes, but through one or two intermediate layers of reactor nodes (RNs) that might serve the same training functions as do the intermediate layers in a neural net: cellular populations could evolve in these reactors that represent the distributed interfaces between the major zones.

While network complexity may not be a problem for zonal replication platforms with a very small number of bioreactors, the Bene network in FIG. 15 is a compact, non-blocking network that could be used as an 8×8 crossbar valve, or even larger networks.

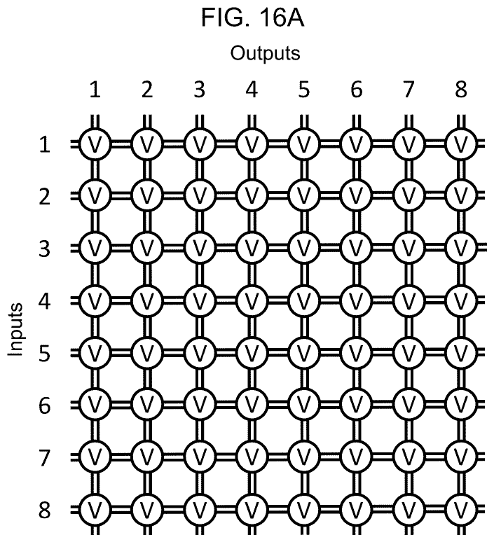

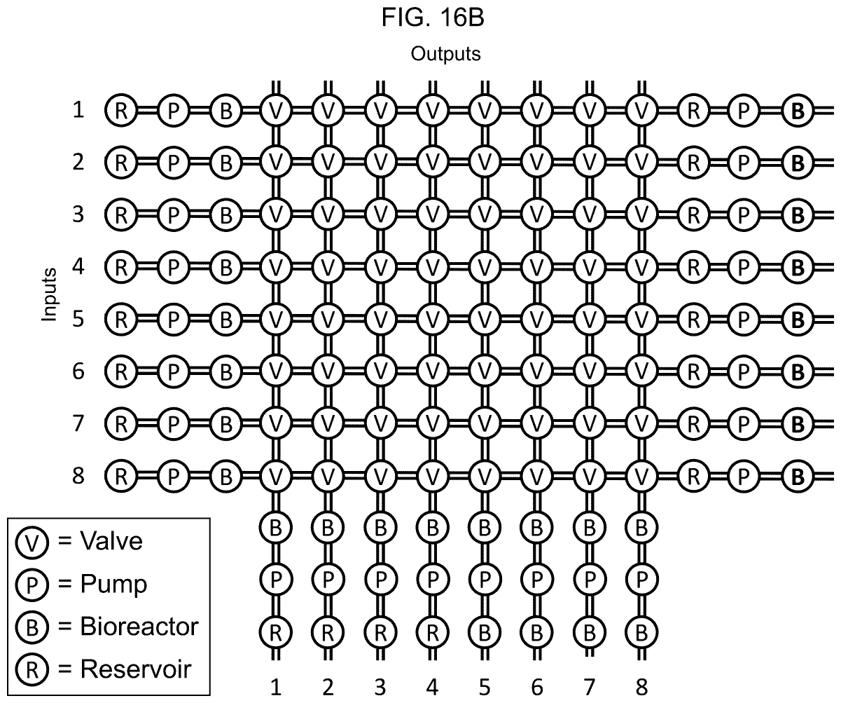

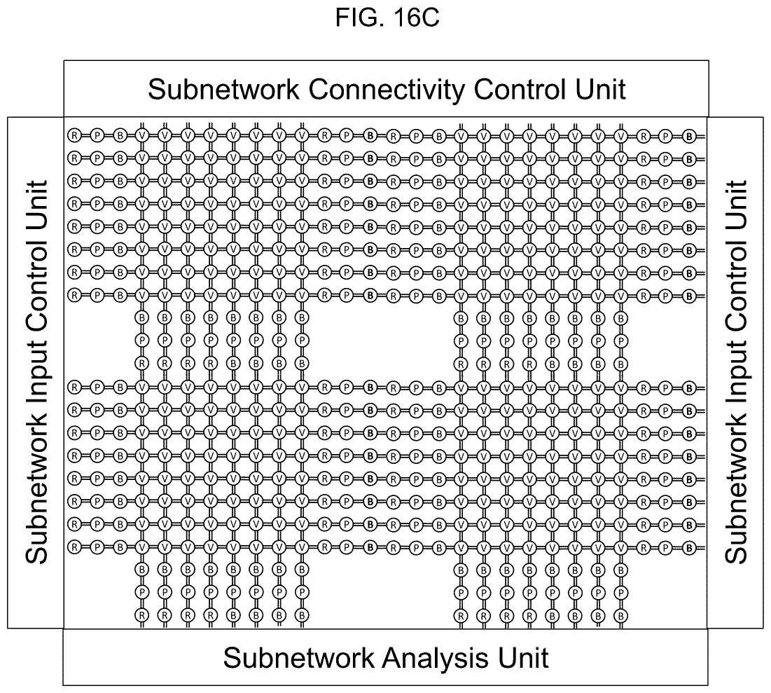

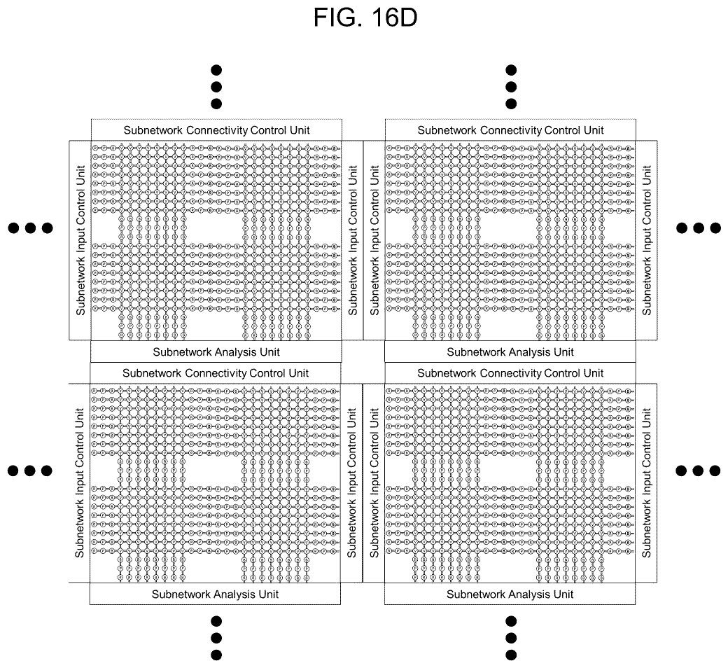

Up to this point, we have been discussing only binary valves that are either open or closed. We now present two embodiments that use simple rotary valves, suitable for either manual or automatic operation, which can provide a smooth gradation in their hydraulic resistance, from full open to full closed. FIG. 16 shows one embodiment of the Simple Grid network in FIG. 12, that could be implemented as a continuously variable, analog 8×8 crossbar valve system that enables continuous transfers between multiple bioreactors, wherein two adjacent half-closed valves V could allow the splitting of an input or output flow between two bioreactors.

FIGS. 16B-16D show how 8×8 simple grid networks could be coupled to bioreactors B, pumps P, and reservoirs R to create fluid handing reactor networks that might be utilized in combinatorial chemical synthesis, synthetic biology, or DNA computing, for example a non-deterministic universal Turing machine implemented with DNA (Currin, A., Korovin, K., Ababi, A., Roper, K., Kell, D., Day, P., and King, R., “Computing exponentially faster: implementing a non-deterministic universal Turing machine using DNA,” Journal of the Royal Society Interface, 14: 20160990 (2017)). Any of the network configurations shown in FIG. 12 through FIG. 16A could be used to create such an integrated network of pumps, valves, reservoirs, reactors and controller, connectivity, and analysis units that would support the transport and mixing of biological or non-biological solutions between reservoirs and analysis of the results of the resulting molecular reactions, for example in combinatorial chemistry wherein aliquots of chemicals are combined or split, or in synthetic biology or DNA computing, wherein aliquots of DNA, RNA, or other molecular sequences are combined or separated. In these cases, the Subnetwork Analysis Units in FIG. 16D could provide a variety of chemical processing steps, in that the network of networks shown in the figure contains modules that can process fluid aliquots. The embodiments shown are not meant to be restrictive, but are simply examples of how the network technologies enabled by this invention might be used for more than just bioreactor zonation modeling.

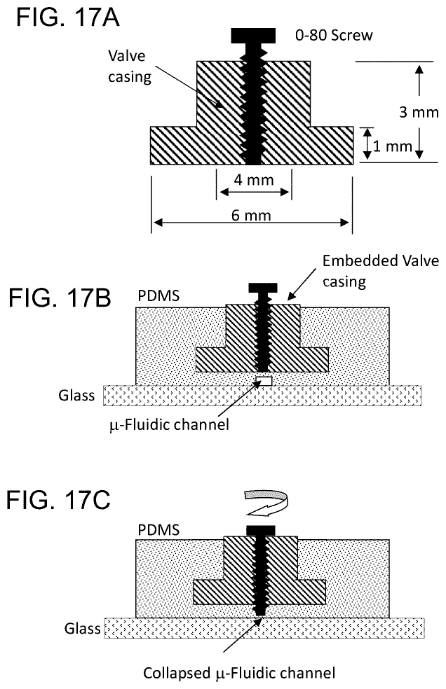

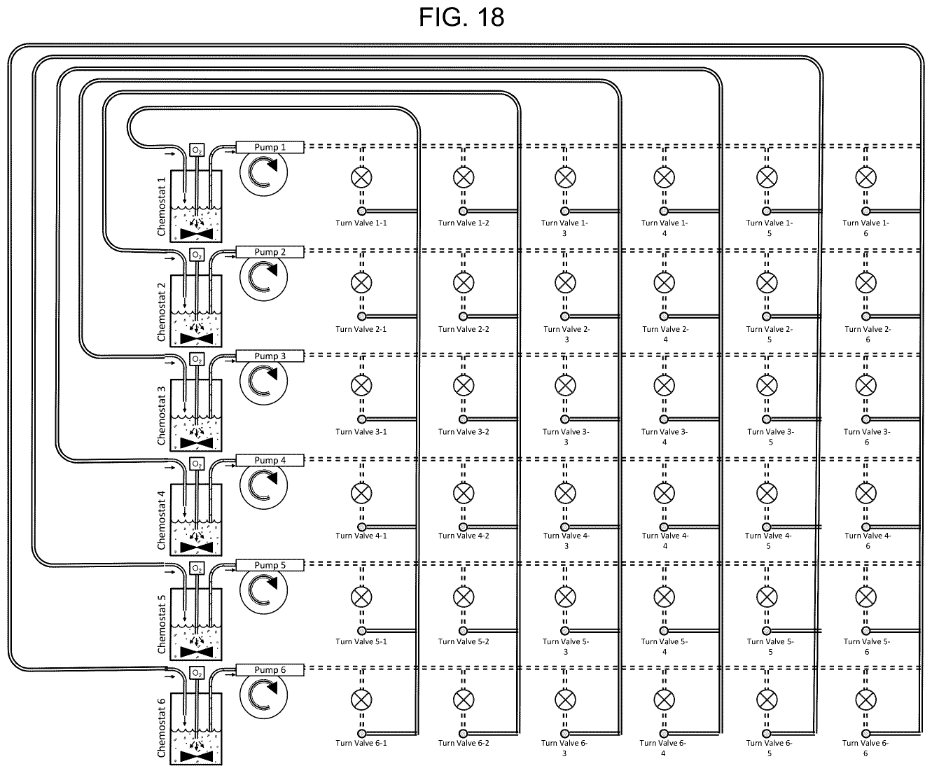

FIG. 17 shows a tape underlayment rotary-node (TURN) valve (Markov, D. A., Manuel, S., Shor, L., Opalenik, S. R., Wikswo, J. P., and Samson, P. C. “Tape underlayment rotary-node (TURN) valves for simple on-chip microfluidic flow control.” Biomedical Microdevices 12(1): 135-144, (2010)). In the exemplary embodiment shown in FIG. 18, a 6×6 crossbar valve system utilizes 36 TURN valves. Each chemostat or bioreactor has an output pump, and the settings of the via valves determine how the effluent from each chemostat or bioreactor is directed to any or all of the six chemostats or bioreactors. The settings of the valves could be adjusted manually, or with computer-controlled motors that could reversibly turn the screws.

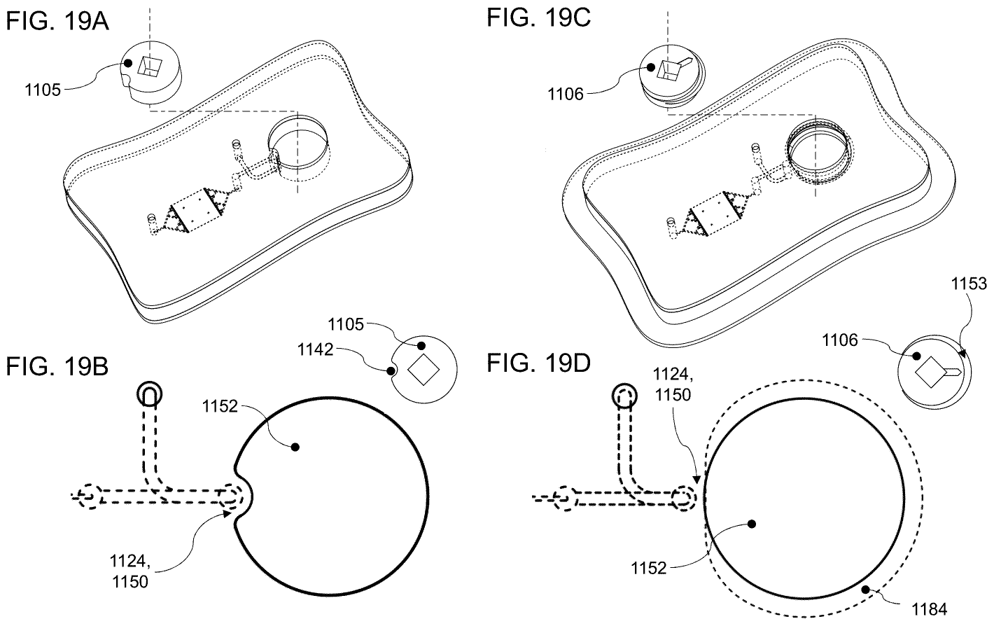

The vertical via valve shown in FIGS. 19A-19D, according to some examples, can be implemented either as on-off (19A and 19B), or variable opening (19C and 19D). The actuators 1106 and 1206 can be rotated either manually or by motors or solenoids, which is disclosed in U.S. patent application Ser. No. 17/917,963, which is incorporated herein by reference in its entirety. One advantage of the via valve over the TURN valve is that overtightening the TURN valve can lead to separation of PDMS layers or tearing the PDMS that is used to create the valved fluidic channels, whereas the strains imposed on the PDMS by the via valve actuator are radial and hence less likely to damage the valve and its surrounding microfluidic channels, and the valve need be actuated only over 360° or less.

### System Configuration (Crossbar Valves)

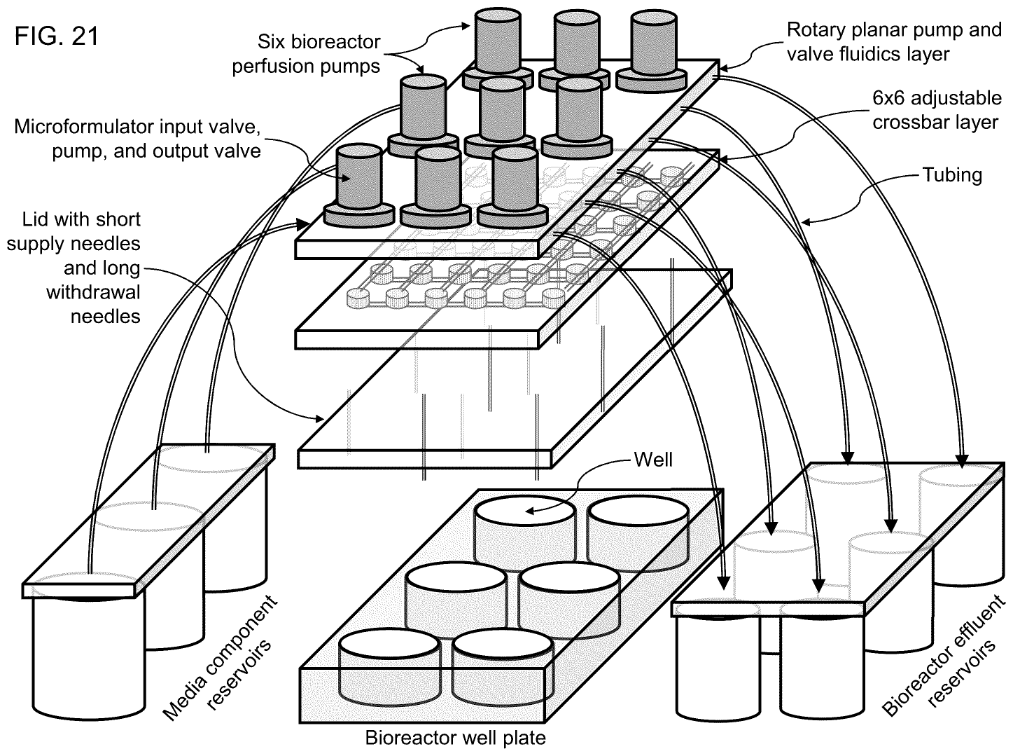

FIG. 21 illustrates one compact implementation of the concepts underlying this invention for experimental study of the effects of bioreactor zonation. In this example, the six wells of a conventional or deep well plate comprise a set of bioreactors, whose stir bars and magnetic stir plate are not shown. Various fluids are drawn from a set of media component reservoirs and are formulated on demand by a microformulator integrated into the system that then delivers the customized solutions to each of the wells by means of the 6×6 adjustable crossbar valve and a set of six short needles attached to the well plate lid. Rotary planar pumps and valves control the rate of perfusion of each well. Longer withdrawal tubes, also connected to the crossbar network, are used to remove fluid and cells from each bioreactor and deliver them to another well or an effluent reservoir. Other physical embodiments can also be used to implement this invention.

### Linear Algebra for Calculating Fluid Distribution

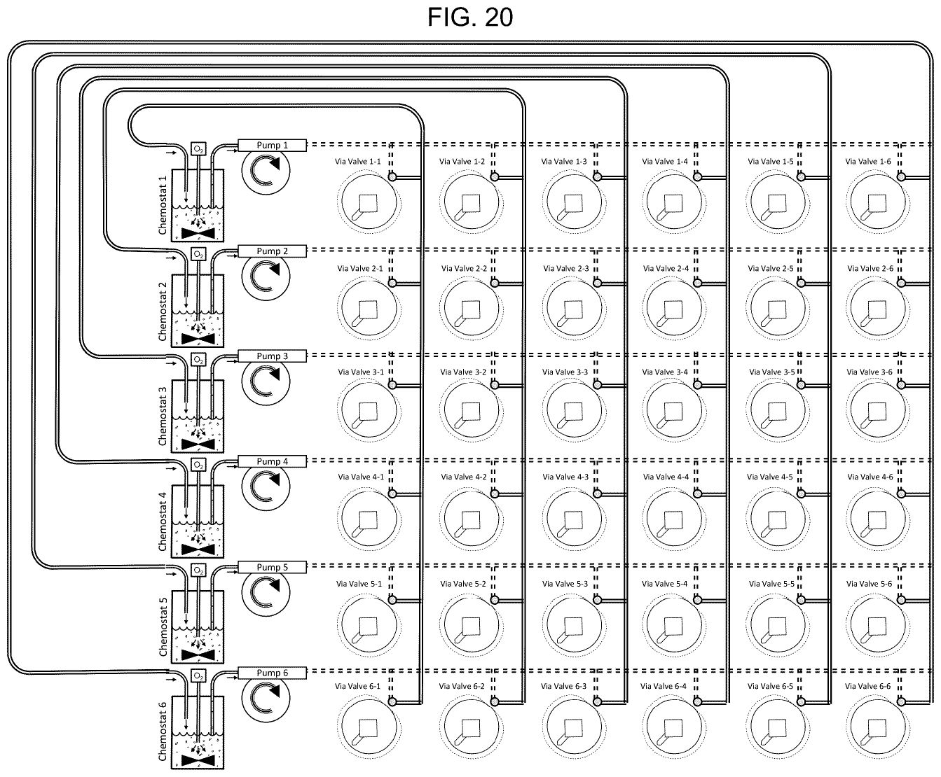

Considering the crossbar networks in shown FIGS. 11A-11E, 16, 18 and 20, the new volume of liquid in each of n bioreactors at time t resulting from the transfer of liquid from one bioreactor to another can be calculated through matrix multiplication:

$$[ \begin{matrix}1 & {dC}_{1} & … & \frac{{dD}_{1}}{{dC}_{n}} \\ \frac{{dD}_{2}}{{dC}_{1}} & 1 & & \\ ⋮ & & ⋱ & \\ \frac{{dD}_{n}}{{dC}_{1}} & … & \frac{{dD}_{n}}{{dC}_{n - 1}} & 1\end{matrix} ] [ \begin{matrix}{C}_{1}^{t - 1} \\ {C}_{2}^{t - 1} \\ ⋮ \\ {C}_{n}^{t - 1}\end{matrix} ] = [ \begin{matrix}{C}_{1}^{t} \\ {C}_{2}^{t} \\ ⋮ \\ {C}_{n}^{t}\end{matrix} ]$$

where Cⱼ^{t-1} is the volume of liquid in bioreactor j (j=1, 2, . . . , n) at time t−1, Cⱼ^{t} is the volume of liquid in bioreactor j at time t, and dCⱼ/dCₖ is the volume of liquid being transferred from bioreactor k to bioreactor j where j≠k and j, k=1, 2, . . . , n. The external removal or addition of liquid into the bioreactors can be calculated using vector addition:

$$[ \begin{matrix}{C}_{1}^{t - 1} \\ {C}_{2}^{t - 1} \\ ⋮ \\ {C}_{n}^{t - 1}\end{matrix} ] + [ \begin{matrix}Δ {C}_{1}^{t} \\ Δ {C}_{2}^{t} \\ ⋮ \\ Δ {C}_{n}^{t}\end{matrix} ] = [ \begin{matrix}{C}_{1}^{t} \\ {C}_{2}^{t} \\ ⋮ \\ {C}_{n}^{t}\end{matrix} ]$$

where ΔCj is the volume of liquid being added or removed from bioreactor j between times t−1 and t.

Equations such as these enable long-term, dynamic computer control of the binary or analog (continuous) crossbar couplings to study dynamic interactions between reactor zones. It is important to realize that the analog or continuous valves shown in FIGS. 17, 19C and 19D allow the division of fluid streams between multiple source and destination reservoirs, so the Boolean logic usually applied to digital electronic gates and crossbars and the vast majority of pneumatic microfluidic valves need not apply to networks whose connectivities are continuous variables described by sets of linear equations such as those represented by the matrix algebra above. This provides important capabilities not only in the bioreactor scale up problem, but also in combinatorial chemical synthesis, synthetic biology, DNA computing, or other operations that could utilize fluidic networks.

### Other Applications

While this invention is described in the context of bioreactors, as suggested above, the principles presented apply equally well to any fluidic system, for example ones used in chemical synthesis or sample purification. For example, the bioreactors could be replaced with a chemical reactor or other physical or chemical processing unit, such that multiple units could be interconnected as desired, as shown in FIG. 16D.

This invention is demonstrated using rotary and pneumatic valves, but any other type of valve could be used as well.

### CONCLUSION

Ideally, a bioreactor is a well-stirred vessel containing a homogenous suspension of cells in media. In practice, large-scale bioreactors have zones with differing cell densities, nutrient and metabolite concentrations, shear forces, and oxygenation. Cells may not thrive in all zones, thereby decreasing the efficiency with which biological products are produced. Scaled-down models must capture phenomena spanning molecules/cells/suspension/zones/reactor. As a result, large-scale bioreactors possess heterogeneous growth conditions that are the result of the limits of mass transfer within the large reactor which may not be evident in laboratory-scale bioreactors. This invention would allow an array of dynamically coupled microchemostats to serve as a physical but smaller scale model of the heterogeneous zonation within a larger reactor, and thereby to support the optimization of cell lines to ensure efficient bioproduction by cells upon scale-up. The embodiments of this invention demonstrate how to construct a fluidic network system that extends to arbitrary variations of the multi-compartment scale-down of bioreactors. Other applications include chemical synthesis, synthetic biology, and DNA computing.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of the invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference were individually incorporated by reference.

### LIST OF REFERENCES

[1]. Dmitry A. Markov, Steven Manuel, Leslie Shor, Susan Opalenik, John P, Wikswo, and Philip C. Samson, “Tape underlayment rotary-node (TURN) valves for simple on-chip microfluidic flow control.” Biomedical Microdevices 12(1): 135-144, (2010)).
[2]. Andrew Currin, Konstantin Korovin, Maria Ababi, Katherine Roper, Douglas B. Kell, Philip J. Day and Ross D. King, “Computing exponentially faster: implementing a non-deterministic universal Turing machine using DNA,” Journal of the Royal Society Interface, 14: 20160990 (2017)).
[3]. Christine Ardal et al. “Antibiotic development—economic, regulatory and societal challenges”. In: Nature Reviews Microbiology 18.5 (2020), pp. 267-274.
[4]. Peter M O'Callaghan et al. “Cell line-specific control of recombinant monoclonal antibody production by CHO cells”. In: Biotechnology and bioengineering 106.6 (2010), pp. 938-951.
[5]. Research ltd and Markets. Biologics Global Market Report 2022: By type, by distribution, by route, by Drug, by mode.
[6]. Bradley W Biggs et al. “Enabling commercial success of industrial biotechnology”. In: Science 374.6575 (2021), pp. 1563-1565.
[7]. Vikramaditya G Yadav et al. “The future of metabolic engineering and synthetic biology: towards a systematic practice”. In: Metabolic engineering 14.3 (2012), pp. 233-241.
[8]. Kerry R Love, Neil C Dalvie, and J Christopher Love. “The yeast stands alone: the future of protein biologic production”. In: Current opinion in biotechnology 53 (2018), pp. 50-58.
[9]. S George, G Larsson, and S—O Enfors. “A scale-down two-compartment reactor with controlled substrate oscillations: metabolic response of Saccharomyces cerevisiae”. In: Bioprocess Engineering 9.6 (1993), pp. 249-257.
[10]. Williams Olughu et al. “Scale-down studies for the scale-up of a recombinant Corynebacterium glutamicum fed-batch fermentation: loss of homogeneity leads to lower levels of cadaverine production”. In: Journal of Chemical Technology & Biotechnology 95.3 (2020), pp. 675-685.
[11]. Alvaro R Lara et al. “Living with heterogeneities in bioreactors”. In: Molecular biotechnology 34.3 (2006), pp. 355-381.
[12]. Gisela Nadal-Rey et al. “Understanding gradients in industrial bioreactors”. In: Biotechnology Advances 46 (2021), p. 107660.
[13]. D W Zabriskie and E J Arcuri. “Factors influencing productivity of fermentations employing recombinant microorganisms”. In: Enzyme and microbial technology 8.12 (1986), pp. 706-717.
[14]. Sudhanshu H Mardikar and Keshavan Niranjan. “Observations on the shear damage to different animal cells in a concentric cylinder viscometer”. In: Biotechnology and Bioengineering 68.6 (2000), pp. 697-704.
[15]. Charles L Cooney. “Bioreactors: design and operation”. In: Science 219.4585 (1983), pp. 728-733.
[16]. Andrew Sparkes et al. “Towards Robot Scientists for autonomous scientific discovery”. In: Automated Experimentation 2.1 (2010), pp. 1-11.
[17]. Ross D King et al. “The robot scientist Adam”. In: Computer 42.8 (2009), pp. 46-54.
[18]. Ronald S Reiserer et al. Continuous automated perfusion culture analysis system (capcas) and applications of same. U.S. patent application Ser. No. 17/578,966. 2022.
[19]. John P Wikswo et al. Multicompartment microfluidic bioreactors, cylindrical rotary valves and applications of same. U.S. patent application Ser. No. 17/057,267. 2021.
[20]. Alvaro R Lara, Laura A Palomares, and Octavio T Ram'irez. “Scale-down: Simulating large-scale cultures in the laboratory”. In: Industrial biotechnology: products and processes (2017), pp. 55-79.
[21]. Tadeusz Iwaniec and Gaven Martin. “The Liouville theorem”. In: Analysis And Topology: A Volume Dedicated to the Memory of S Stoilow. World Scientific, 1998, pp. 339-361.
[22]. Peter Neubauer and Stefan Junne. “Scale-down simulators for metabolic analysis of large-scale bioprocesses”. In: Current opinion in biotechnology 21.1 (2010), pp. 114-121.
[23]. Andrew Hall Cutler, Michael Jerry Antal Jr, and Maitland Jones Jr. “A critical evaluation of the plug-flow idealization of tubular-flow reactor data”. In: Industrial & engineering chemistry research 27.4 (1988), pp. 691-697.
[24]. Feng Li et al. “A systematic approach for scale-down model development and characterization of commercial cell culture processes”. In: Biotechnology progress 22.3 (2006), pp. 696-703.
[25]. Peter Neubauer and Stefan Junne. Scale-up and scale-down methodologies for bioreactors. Chichester: John Wiley & Sons, 2016.
[26]. Guan Wang et al. “Developing a computational framework to advance bioprocess scale-up”. In: Trends in Biotechnology 38.8 (2020), pp. 846-856.
[27]. John David Anderson and J Wendt. Computational fluid dynamics. Vol. 206. Springer, 1995.
[28]. Zuoliang Sha, Arto Laari, and Ilkka Turunen. “Multi-Phase-Multi-Size-Group Model for the Inclusion of Population Balances into the CFD Simulation of Gas-Liquid Bubbly Flows”. In: Chemical Engineering & Technology: Industrial Chemistry-Plant Equipment-Process Engineering-Biotechnology 29.5 (2006), pp. 550-559.
[29]. Maxime Moreau et al. “A new numerical method for axial dispersion characterization in microreactors”. In: Chemical Engineering Science 168 (2017), pp. 178-188.
[30]. A Moser. “Tubular fermenter for aerobic processes. I: Physical characteristics”. In: Biotech. Bioeng. Symposium. Vol. 4. 1973, pp. 399-411.
[31]. H Singh and D W Hutmacher. “Bioreactor studies and computational fluid dynamics”. In: Bioreactor Systems for Tissue Engineering (2009), pp. 231-249.
[32]. Jeffrey D Orth, Ines Thiele, and Bernhard Ø Palsson. “What is flux balance analysis?” In: Nature biotechnology 28.3 (2010), pp. 245-248.
[33]. Federico Sporleder et al. “On the population balance equation”. In: Reviews in Chemical Engineering 28.2-3 (2012), pp. 149-169.
[34]. Kenneth J Kauffman, Purusharth Prakash, and Jeremy S Edwards. “Advances in flux balance analysis”. In: Current opinion in biotechnology 14.5 (2003), pp. 491-496.
[35]. Frank R Schmidt. “Optimization and scale up of industrial fermentation processes”. In: Applied microbiology and biotechnology 68.4 (2005), pp. 425-435.

What is claimed is:

1. A network platform, comprising:
a fluidic network comprising one or more pumps, and one or more valves; and
a plurality of fluidic modules interconnected by the fluidic network of the one or more pumps and the one or more valves to allow controlled transfer of suspended cells and fluids from one fluidic module to another, or self-circulating.

2. The network platform of claim 1, wherein the plurality of fluidic modules comprises bioreactors, wells, organs-on-chips, chemostats, or a combination of them.

3. The network platform of claim 1, further comprising at least one input media reservoir and/or at least one collection reservoir in fluidic communication with the fluidic network for providing inputs and/or collecting outputs of any one of the plurality of fluidic modules, respectively.

4. The network platform of claim 3, wherein each of the plurality of fluidic modules is individually perfusable.

5. The network platform of claim 4, wherein a rate of perfusion of each fluidic module is controlled by at least one of the one or more pumps and the one or more valves.

6. The network platform of claim 1, wherein the one or more valves are a rotary planar valve system that is operably regulated with a single motor as a time-domain fluidic multiplexer to move samples between each and every one of the plurality of fluidic modules.

7. The network platform of claim 1, wherein the one or more valves comprise an N×M crossbar valve that operably connects the at least one input media reservoir to the inputs of any one of the plurality of fluidic modules, and the outputs of the plurality of bioreactors to either the input of one of the bioreactors or the at least one collection reservoir, wherein each of N and M is an integer greater than zero.

8. The network platform of claim 1, wherein the one or more valves comprise n two-state crossbar valves, thereby creating 2ⁿ possible valve states, wherein n is an integer greater than zero.

9. The network platform of claim 1, wherein the fluidic network is a continuously pumped fluidic network configured to ensure that living cells are in tubes for only very short intervals of time, by being moved directly from one bioreactor to another without intermediate storage.

10. The network platform of claim 9, wherein the one or more pumps and the one or more valves are configured to ensure that all fluid lines are promptly washed to avoid the trapping or storage of cells in sub-optimal environments.

11. The network platform of claim 9, wherein the fluidic network further comprises a separating means coupled with the one or more pumps and the one or more valves for separating cells such that certain cells are recirculated to one fluidic module while others are allowed to be moved to another.

12. The network platform of claim 11, wherein the separating means comprises a filter or other means to retain all cells within a fluidic module and only extract the fluid from one fluidic module for transfer to another.

13. The network platform of claim 11, wherein the separating means comprises a tangential flow filter, an alternating tangential flow filter, spiral cell separators, or other means.

14. The network platform of claim 1, wherein the fluidic network is a single large-scale crossbar valve system that operates with a single pneumatic, vertical via, rotary, or other mechanical valve at each intersection between every fluidic module inflow and outflow line, with a pump on each of either the inflow or outflow lines, or a dynamic multi-stage interconnection network that uses multiple smaller-scale crossbar or other valves.

15. The network platform of claim 1, wherein the fluidic network is a dynamically reconfigurable network.

16. The network platform of claim 1, wherein the interconnections of the plurality of fluidic modules are configured to create or simulate biological systems in which there are large-scale spatial gradients that support a variation in microbial composition.

17. The network platform of claim 1, wherein the interconnections of the plurality of fluidic modules are configured to allow any or all of the plurality of fluidic modules to connect to any other or all of the other fluidic modules.

18. The network platform of claim 1, wherein the plurality of fluidic modules is configured to serve as a physical but smaller scale model of the heterogeneous zonation within a larger reactor, and thereby to support the optimization of cell lines to ensure efficient bioproduction by cells upon scale-up.

19. The network platform of claim 1, wherein the plurality of fluidic modules comprises multiple microbioreactors that are linked together into a single combined bioreactor system to operably simulate the traversal of a cell through the different zonal conditions of the industrial bioreactor, creating a small-scale system that is able to model the conditions within an industrial-scale bioreactor.

20. The network platform of claim 1, wherein the plurality of fluidic modules comprises multiple microbioreactors configured to simulate spatiotemporal heterogeneities that are inherent in large-scale bioreactors, such that each microbioreactor represents a region or zone with a set of cell culture parameters including cell density, cell replication rate and division state, pH, shear stress, temperature mixing rates, and the concentration of nutrients, metabolites, oxygen, carbon dioxide, and other gases.

21. The network platform of claim 1, being usable in combinatorial chemical processing, in which aliquots of different chemicals are combined or split.

22. The network platform of claim 1, being usable in synthetic biology and/or DNA computing, in which aliquots of specifically coded RNA or DNA, or other molecular sequences are combined or separated.
